SEQUENCE LISTING (38 sequences)

SEQ ID NO: 1 - DNA, 21 bp, Artificial Sequence
  forward primer for genotyping the ANT1 gene
ggaaggacagctaacgatgtg

SEQ ID NO: 2 - DNA, 22 bp, Artificial Sequence
  reverse primer for genotyping the ANT1 gene
gttgcatgggtggtaaattaag

SEQ ID NO: 3 - DNA, 22 bp, Artificial Sequence
  forward primer for genotyping the CHS1 gene
tgagatcactgcagttacgttc

SEQ ID NO: 4 - DNA, 19 bp, Artificial Sequence
  reverse primer for genotyping the CHS1 gene
taagcccagcccactaagc

SEQ ID NO: 5 - DNA, 19 bp, Artificial Sequence
  forward primer for genotyping the CHS2 gene
tctgcagcccaaactctac

SEQ ID NO: 6 - DNA, 23 bp, Artificial Sequence
  reverse primer for genotyping the CHS2 gene
tatggagcacaacagtctcaaca

SEQ ID NO: 7 - DNA, 21 bp, Artificial Sequence
  forward primer for genotyping the DFR gene
gataaggacttggccctagtg

SEQ ID NO: 8 - DNA, 19 bp, Artificial Sequence
  reverse primer for genotyping the DFR gene
gatacgcgagagccttcag

SEQ ID NO: 9 - DNA, 22 bp, Artificial Sequence
  forward primer for genotyping the F3H gene
ccaatcaaagacgcattagcag

SEQ ID NO: 10 - DNA, 23 bp, Artificial Sequence
  reverse primer for genotyping the F3H gene
tcacaaggaaggccaagataaag

SEQ ID NO: 11 - DNA, 22 bp, Artificial Sequence
  forward primer for real-time PCR analysis of the CHS1 gene
gcctctacaaaagaaggcctag

SEQ ID NO: 12 - DNA, 19 bp, Artificial Sequence
  reverse primer for real-time PCR analysis of the CHS1 gene
taagcccagcccactaagc

SEQ ID NO: 13 - DNA, 23 bp, Artificial Sequence
  forward primer for real-time PCR analysis of the CHS2 gene
catccaaagaagggcttagtacc

SEQ ID NO: 14 - DNA, 23 bp, Artificial Sequence
  reverse primer for real-time PCR analysis of the CHS2 gene
tatggagcacaacagtctcaaca

SEQ ID NO: 15 - DNA, 21 bp, Artificial Sequence
  forward primer for real-time PCR analysis of the CHI gene
tttcaaggcttccaggatatg

SEQ ID NO: 16 - DNA, 20 bp, Artificial Sequence
  reverse primer for real-time PCR analysis of the CHI gene
atgtcccgaacttctccttg

SEQ ID NO: 17 - DNA, 20 bp, Artificial Sequence
  forward primer for real-time PCR analysis of the DFR gene
ttcaagtggcaaggagaatg

SEQ ID NO: 18 - DNA, 23 bp, Artificial Sequence
  reverse primer for real-time PCR analysis of the DFR gene
agaacatgttggtgaggtagctc

SEQ ID NO: 19 - DNA, 22 bp, Artificial Sequence
  forward primer for real-time PCR analysis of the F3H gene
gtgaatcctagccttgacagtg

SEQ ID NO: 20 - DNA, 22 bp, Artificial Sequence
  reverse primer for real-time PCR analysis of the F3H gene
gctttaccagaaggcctcttac

SEQ ID NO: 21 - DNA, 22 bp, Artificial Sequence
  forward primer for real-time PCR analysis of the ANT1 gene
gacgcaagtatttctcaagcac

SEQ ID NO: 22 - DNA, 20 bp, Artificial Sequence
  reverse primer for real-time PCR analysis of the ANT1 gene
tccaccatggatctacgttg

SEQ ID NO: 23 - DNA, 21 bp, Artificial Sequence
  forward primer for real-time PCR analysis of 18s ribosomal RNA
gcgacgcatcattcaaatttc

SEQ ID NO: 24 - DNA, 21 bp, Artificial Sequence
  reverse primer for real-time PCR analysis of 18s ribosomal RNA
tccggaatcgaaccctaattc

SEQ ID NO: 25 - DNA, 27 bp, Artificial Sequence
  forward primer for mapping the AFT gene to the tomato genome
tcccccgggatgaacagtacatctatg

SEQ ID NO: 26 - DNA, 33 bp, Artificial Sequence
  reverse primer for mapping the AFT gene to the tomato genome
ggactagtttaatcaagtagattccataagtca

SEQ ID NO: 27 - DNA, 28 bp, Solanum lycopersicum
  misc_feature (1)..(28): 5′ coding sequence of the ANT1 gene, strains Ailsa Craig and LA1996
atgaacagtacatctatgtcttcattgg

SEQ ID NO: 28 - DNA, 32 bp, Solanum lycopersicum
  misc_feature (1)..(32): 3′ coding sequence of the ANT1 gene, strains Ailsa Craig and LA1996
ggactagtttaatcaagtagattccataagtc

SEQ ID NO: 29 - DNA, 1013 bp, Solanum lycopersicum
  misc_feature (1)..(1013): ANT1 gene, strain Ailsa Craig
atgaacagtacatctatgtcttcattgggagtgagaaaaggttcatggactgatgaagaagattttcttctaagaaaatgtattgataagtatggtgaaggaaaatggcatcttgttcccataagagctggtaacctattaaattaactatcacgttatttttatttgtctttctgtctcattttatttgacgttattacgaatatcatctgaaaatgtacgtgcaggtctgaatagatgtcggaaaagttgtagattgaggtggctgaattatctaaggccacatatcaagagaggtgactttgaacaagatgaagtggatctcattttgaggcttcataagctcttaggcaacaggcatgcaagtttatgttttgacaaaatttgattagtatatattatatatacgtgtgactatttcatctaaatgttacgttattttaggtagatggtcacttattgctggtagacttcccggaaggacagctaacgatgtgaaaaactattggaacactaatcttctaaggaagttaaatactactaaaattgttcctcgcgaaaagattaacaataagtgtggagaaattagtactaagattgaaattataaaacctcaacgacgcaagtatttctcaagcacaatgaagaatgttacaaacaataatgtaattttggacgaggaggaacattgcaaggaaataataagtgagaaacaaactccagatgcatcgatggacaacgtagatccatggtggataaatttactggaaaattgcaatgacgatattgaagaagatgaagaggttgtaattaattatgaaaaaacactaacaagtttgttacatgaagaaatatcaccaccattaaatattggtgaaggtaactccatgcaacaaggacaaataagtcatgaaaattggggtgaattttctcttaatttaccacccatgcaacaaggagtacaaaatgatgatttttctgctgaaattgacttatggaatctacttgattaa

SEQ ID NO: 30 - DNA, 1008 bp, Solanum lycopersicum
  misc_feature (1)..(1008): ANT1 gene, strain LA1996
atgaacagtacatctatgtcttcattgggagtgagaaaaggttcatggactgatgaagaagattttcttttaagaaaatgtattgataagtatggtgaaggaaaatggcatcttgttcccataagagctggtaattattaaactgactatcacgttatttttatctgtctgtctcattttatatgacgttattttgaacattatctgaaaatgtacgtgcaggtctgaatagatgtcggaaaagttgtagattgaggtggctgaattatctaaggccacatatcaagagaggtgactttgaacaagatgaagtggatctcattttgaggcttcataagctcttaggcaacaggcatgcaagtttatgttttgacaaaatttgattagtatatattatatatacgtgtgactatttcatctaaatgttacattattttacgtagatggtcacttattgctggtagacttccaggaaggacagctaacgatgtgaaaaactattggaacactaatcttctaaggaagttaaatactactaaaattgttcctcgtgaaaagactaacaataagtgtggagaaattagtactaagattgaaattataaaacctcaaccacgaaagtatttctcaagcacaatgaagaatattacaaacaatattgtaattttggacgaggaggaacattgcaaggaaataaaaagtgagaaacaaactccagatgcatcgatggacaacgtagatcaatggtggataaatttactggaaaattgcaatgacgatattgaagaagatgaagaggttgtaattaattatgaaaaaacactaacaagtttgttacatgaagaaaaatcaccaccattaaatattggtgaaggtaactccatgcaacaaggacaaataagtcatgaaaattggggtgaattttctcttaatttacaacccatgcaacaaggagtacaaaatgatgatttttctgctgaaattgacttatggaatctacttgattaa

SEQ ID NO: 31 - protein, 274 aa, Solanum lycopersicum
  UNSURE (1)..(274): ANT1 protein, strain Ailsa Craig
MNSTSMSSLGVRKGSWTDEEDFLLRKCIDKYGEGKWHLVPIRAGLNRCRKSCRLRWLNYLRPHIKRGDFEQDEVDLILRLHKLLGNRWSLIAGRLPGRTANDVKNYWNTNLLRKLNTTKIVPREKINNKCGEISTKIEIIKPQRRKYFSSTMKNVTNNNVILDEEEHCKEIISEKQTPDASMDNVDPWWINLLENCNDDIEEDEEVVINYEKTLTSLLHEEISPPLNIGEGNSMQQGQISHENWGEFSLNLPPMQQGVQNDDFSAEIDLWNLLD

SEQ ID NO: 32 - protein, 274 aa, Solanum lycopersicum
  UNSURE (1)..(274): ANT1 protein, strain LA1996
MNSTSMSSLGVRKGSWTDEEDFLLRKCIDKYGEGKWHLVPIRAGLNRCRKSCRLRWLNYLRPHIKRGDFEQDEVDLILRLHKLLGNRWSLIAGRLPGRTANDVKNYWNTNLLRKLNTTKIVPREKTNNKCGEISTKIEIIKPQPRKYFSSTMKNITNNIVILDEEEHCKEIKSEKQTPDASMDNVDQWWINLLENCNDDIEEDEEVVINYEKTLTSLLHEEKSPPLNIGEGNSMQQGQISHENWGEFSLNLQPMQQGVQNDDFSAEIDLWNLLD

SEQ ID NO: 33 - protein, 274 aa, Solanum lycopersicum
  UNSURE (1)..(274): ANT1 protein, strain PI128650
MNSTSMSSLGVRKGSWADEEDFLLRKCIDKYGEGKWHLVPIRAGLNRCRKSCRLRWLNYLRPHIKRGDFEQDEVDLILRLRKLLGTRWSLIAGRLPGRTANDVKNYWNTNLLRKLNTTKIVPREKINIKCGEISTKIEIIKPQPRKYFSSTMKNVTNNIVILDEEEHCKEIISEKQTPDASMDNVDQWWINLLENCNDGIEEDEEVVINYEKTLTSLLHEEISPPLNIGEGNSMQQGQISHENWGEFSLNLPPMQQGVQNDDFSAEIDLWNLLD

SEQ ID NO: 34 - protein, 274 aa, Solanum lycopersicum
  UNSURE (1)..(274): ANT1 protein, strain hp-799
MNSTSMSSLGVRKGSWADEEDFLLRKCIDKYGEGKWHLVPIRAGLNRCRKSCRLRWLNYLRPHIKRGDFEQDEVDLILRLHKLLGNRWSLIAGRLPGRTANDVKNYWNTNLLRKLNTTKIVPREKINIKCGEISTKIEIIKPQPRKYFSSTMKNVTNNIVILDEEEHCKEIISEKQTPDASMDNVDQWWINLLENCNDGIEEDEEVVINYEKTLTSLLHEEISPPLNIGEGNSMQQGQISHENWGEFSLNLPPMQQGVQNDDFSAEIDLWNLLD

SEQ ID NO: 35 - protein, 274 aa, Solanum lycopersicum
  UNSURE (1)..(274): ANT1 protein, strain LA1589
MNSTSMSSLGVRKGSWTDEEDFLLRKCIDKYGEGKWHLVPIRAGLNRCRKSCRLRWLNYLRPHIKRGDFEQDEVDLILRLHKLLGNRWSLIAGRLPGRTANDVKNYWNTNLLRKLNTTKIVPREKINNKCGEISTKIEIIKPQPRKYFSSTMKNVTNNNVILDEEEHCKEIISEKQTPDASMDNVDPWWINLLENCNDDIEEDEEVVINYEKTLTSLLHEEISPPLNIGEGNSMQQGQISHENWGEFSLNLPPMQQGVQNDDFSAEIDLWNLLD

SEQ ID NO: 36 - protein, 274 aa, Solanum lycopersicum; identical to SEQ ID NO: 31
  UNSURE (1)..(274): ANT1 protein, strain LA2838A

SEQ ID NO: 37 - protein, 274 aa, Solanum lycopersicum; identical to SEQ ID NO: 32
  UNSURE (1)..(274): ANT1 protein, strain LA1996

SEQ ID NO: 38 - protein, 262 aa, Solanum lycopersicum
  UNSURE (1)..(262): ANT1 protein, strain CAE75745
MNTAIIAKSSGVRKGAWTEEEDFLLRKCIQNYGEGKWHLVPIRAGLNRCRKSCRLRWLNYLRPHIKRGDFGWDEIDLILRLHKLLGNRWSLIAGRLPGRTANDVKNYWNSHLQKKLITAPHRQEKKYNTALKITTKNVLRPRPRTFSSSAKNNISWCTNKSTVITNTLDKDERDKEIGLNICQKLTSETSSTIDDGVQWWTSLLENCKEIEEDVAAVGIFEEKNKLVPSLLHDEINSLTMQQGQSDGWDDFSADIDLWNLLN

(19) United States
(12) Patent Application Publication
(10) Pub. No.: US 2010/0199370 A1
(43) Pub. Date: Aug. 5, 2010

(54) MEANS AND METHODS OF PRODUCING FRUITS WITH HIGH LEVELS OF ANTHOCYANINS AND FLAVONOLS

(75) Inventors: Ilan Levin, Mazkeret Batia (IL); Michal Oren-Shamir, Rehovot (IL); Maya Sapir, Rehovot (IL); Moshe Reuveni, Mevasseret Zion (IL); Yaakov Tadmor, Tamrat (IL)

Correspondence Address: Fleit Gibbons Gutman Bongini & Bianco PL, 21355 EAST DIXIE HIGHWAY, SUITE 115, MIAMI, FL, 33180, US

(73) Assignees: THE STATE OF ISRAEL, MINISTRY OF AGRICULTURE & RURAL DEVELOPMENT, AGRICULTURAL RESEARCH; ORGANIZATION (A.R.O.) , THE VOLCANI CENTER, Beit-Dagan (IL)

(21) Appl. No.: 12/526,173

(22) PCT Filed: Feb. 6, 2008
(86) PCT No.: PCT/IL2008/000159

§ 371 Date: Apr. 5, 2010

(30) Foreign Application Priority Data
Feb. 6, 2007 (IL) 181193

(51) Int. Cl. A01H 5/00; C12N 15/82; A01H 1/02; C07H 21/04; C12Q 1/68

(52) U.S. Cl. 800/260; 800/317.4; 800/298; 800/278; 536/23.6; 435/6

(57) ABSTRACT

The invention includes means and methods for providing an AFT gene encoding a protein characterized by at least 80% identity with the amino acid sequence shown in FIG. 9 (LA1996; SEQ ID NO:37) having been genetically introgressed into cultivated tomato plants or elite lines. The AFT gene confers higher concentrations of flavonoids to the plants compared with prior art cultivated plants that were not introgressed with the gene. An AFT S. chilense genotype introgressively-derived tomato plant is disclosed. Transgenic plants expressing metabolites of the flavonoid pathway, especially anthocyanin or flavonols, in plants, plant parts or seeds thereof, carrying particular DNA sequences recombinable into a plurality of one or more transformation and/or expression vectors, useful for transformation and/or expression in plants are disclosed. Methods of obtaining same are disclosed.

### FIELD OF THE INVENTION

The present invention generally relates to means and methods of producing fruits, especially tomato fruits, with high anthocyanins, especially delphinidin, petunidin and malvidin and high flavonol phenotypes, especially quercetin and kaempherol.

### BACKGROUND OF THE INVENTION

Enriching fruits and vegetables with functional metabolites such as carotenoid, flavonoids and vitamins has become an important breeding goal in the past few years. A good example of the trend is the introgression of the high pigment (hp) mutations into commercial tomato cultivars in order to enrich their fruits with higher levels of carotenoids, flavonoids and vitamins C and E.

The ANTHOCYANIN FRUIT (AFT) genotype, originating from S. chilense, is characterized by purple color in skin and outer pericarp tissues of its fruits, due to high levels of anthocyanins, metabolites that belong to the flavonoids family. It was reported that this increase in anthocyanin levels is determined by a single gene (Jones et al., (2003) J Hered. 94, 449-446). Flavonoids are polyphenolic compounds that occur naturally in most plants. Flavonoids are present in fruits, vegetables and beverages derived from plants (tea, red wine), and in many dietary supplements or herbal remedies. Based on their core structure, the aglycone, they can be grouped into different classes, such as chalcones, flavanones, dihydroflavonols, flavonols, and anthocyanins. To date, more than 4000 different flavonoids have been identified. This large diversity is attributable to single or combinatorial modifications of the aglycone, such as glycosylation, methylation, and acylation. As a group, flavonoids are involved in many aspects of plant growth and development, such as pathogen resistance, pigment production, UV light protection, pollen growth, and seed coat development (Harborne, (1986) The Flavonoids. Advances in Research Since 1986, 1st ed; (Bovy et al., (2002), Plant Cell 14, 2509-2526).

Anthocyanins are the most common class of purple, red, and blue plant pigments. More than 300 different anthocyanin compounds have been identified in plants. They are planar molecules with a C6-C3-C6 carbon structure typical of flavonoids.

There is increasing evidence to suggest that flavonoids, in particular those belonging to the class of flavonols (such as kaempferol and quercetin), are potentially health-protecting components in the human diet as a result of their high antioxidant capacity (Rice Evans et al., (1997), Trends Plant Sci 2: 152-159), (Proteggente et al., (2002), Free Radic. Res 36: 217-233) and their ability, in vitro, to induce human protective enzyme systems (Cook and Samman, (1996) J. Nutr Biochem 7, 66-76). Based on these findings, it was postulated that flavonoids may offer protection against major diseases such as coronary heart disease and cancer (Hertog and Holtman, (1996) Eur J Clin Nutr 50, 63-71). Several epidemiological studies have suggested a direct relationship between cardioprotection and consumption of flavonols from dietary sources such as onion, apple, and tea (Hertog et al., (1993) Lancet 342: 1007-1011). In this respect anthocyanins have received particular attention because of their very strong antioxidant activity as measured by the oxygen radical absorbing capacity (ORAC) assay. Grapes (Wang et al., (1996) J Agric Food Chem 44: 701-705), blueberries, blackberries, raspberries, and cherries (Wang et al., (1997) J Agric Food Chem 45: 304-309) are known to contain high levels of anthocyanins, and share high antioxidant capacity in comparison to other fruits and vegetables. Tomato, being one of the most important food crops worldwide and generally more affordable and widely consumed than grapes, berries and cherries, could serve as a better candidate for use as a source for anthocyanin consumption. However commercially available tomatoes are not characterized by particularly high concentrations of flavonoids (including anthocyanins), rendering the realization of a commercial tomato plant with high levels of flavonoids an important goal.

Strategies to increase and diversify the content of flavonoids in tomato fruits focus mainly on:
1. metabolomic engineering of structural genes involved in flavonoids biosynthesis;
2. transgenic modulation of transcription factors affecting these metabolic pathways; and
3. single-point mutations (spontaneous or induced), and/or quantitative trait loci with pronounced effects on such phytonutrient levels (Levin et al., (2006) Israel J of Plant Sci, in press).

Transformation of tomato plants with the CHALCONE ISOMERASE (CHI) gene from P. hybrida, under the control of the 35S promoter, resulted in a dramatic increase in peel flavonol levels. However, no increase in flavonol levels were observed in leaves and in green, breaker and turning tomato flesh from high flavonol transgenic plants (Muir et al., (2001) Nat Biotechnol 19: 470-474).

Flavonol accumulation in tomato flesh, and hence an overall increase in flavonoid levels in tomato fruit, was achieved by simultaneous overexpression of the maize genes encoding the transcription factors LC and C1 (Bovy et al., (2002) Plant Cell 14: 2509-2526).

In an alternative approach, genes encoding four key biosynthetic enzymes from P. hybrida leading to flavonols: CHALCONE SYNTHASE (CHS), CHALCONE SYNTHASE (CHI), FLAVANONE-3-HYDROXYLASE (F3H), and FLAVONOL SYNTHASE (FLS) were ectopically and simultaneously expressed in tomato plants. About 75% of the primary transformants containing all four transgenes accumulated very high levels of quercetin glycosides in the peel and, more modest, but significantly increased levels of kaempferol and naringenin-glycosides in columella tissue (Verhoeyen et al. (2002) J Exp Bot 53: 2099-2106).

It can be noted that overall high flavonoid content in tomato fruit flesh by transgenic genetic modification has not been highly successful. In addition, there is an increasing demand for alternative non-transgenic approaches for achieving high flavonoids. This demand is motivated by consumers' reluctance to consume transgenic fruits and vegetables, also known as genetically modified organisms (GMO).

The fruit of several tomato species related to the cultivated tomato; S. chilense, S. habrochaites, S. cheesmaniae, and S. lycopersicoides contain significantly higher amounts of anthocyanins (Rick, (1964) Occas Paper Calif Acad Sci 44: 59; Giorgiev, (1972) Rep Tomato Genet Coop 22:10; Rick et al., (1994) Rep. Tomato Genet Coop 44: 29-30). ANTHOCYANIN FRUIT (AFT) from S. chilense, AUBERGINE (ABG) from S. lycopersicoides, and atroviolacium (atv) from L. cheesmaniae cause anthocyanin expression in tomato fruit. The wild species S. pennellii v. puberulum was shown to be a source for enriching tomato fruits with functional flavonoids (Willits et al., (2005) J Agric Food Chem 53: 1231-1236) but the pericarpal concentrations were modest and the progeny were unstable.

Another approach of increasing fruit flavonoids is through the introgression of high pigment (hp) mutations. Tomato hp mutations (hp-1, hp-1^{w}, hp-2, hp-2^{j}, hp-2^{dg}) are best known for their positive effect on carotenoid (lycopene and carotenes) levels in ripe red fruits (Levin et al., (2003) Theor Appl Genet. 106, 454-460). Mature fruits of plants carrying the hp-1 mutation were also found to exhibit a 13-fold increase of the flavonoid quercetin in tomato fruit pericarp relative to their isogenic counterparts (Yen et al., (1997) Theor. Appl. Genet. 95: 1069-1079). Similar increases in quercetin levels in fruits of the tomato mutant hp-2^{dg} and in the fruit peel of the tomato mutants hp-2 and hp-2^{j} were also noted (Bino et al., (2005) New Phytologist 166, 427-438; Levin et al., (2006) Israel J of Plant Sci, in press).

S. chilense fruits of the AFT genotype are characterized by anthocyanin in the skin and outer pericarp tissues of the fruit. Segregation ratios of anthocyanin expression in F₂ and BC₁ populations of a cross between processing tomatoes and AFT plants were found to be consistent with a single dominant gene hypothesis for anthocyanin expression. T-DNA activation-tagging experiments in tomato fruits identified a MYB transcriptional regulator of anthocyanin biosynthesis, termed ANT1 that has high homology with Petunia An2 (Mathews et al., (2003) Plant Cell 15: 1689-1703).

Mutant ant1 tomato plants showed intense purple pigmentation from the very early stage of shoot formation in culture, reflecting activation of the biosynthetic pathway leading to anthocyanin accumulation. Vegetative tissues of ant1 plants displayed intense purple color; however, the fruit only showed purple spotting on the epidermis that could be visualized only under X66 magnification. It is therefore a long felt need to provide high anthocyanin tomato plants with higher concentrations of anthocyanins on the epidermis and outer pericarp, such that the phenotype is more intensely purple.

Since AFT, derived from a wild S. chilense tomato strain, provides high anthocyanin concentrations, and hp-1 commercial tomatoes were shown to have some enhancement in particular flavonoid concentrations, a stably breeding accession derived therefrom which had strongly enhanced flavenoid concentrations would usefully fulfill a long felt need.

The AFT S. chilense gene is known to be responsible for higher anthocyanin concentrations than the cultivated tomato counterpart. Therefore the characterization, isolation and transformation of this gene into commercial plants including tomato, such that flavenoid concentrations were enhanced, would again fulfill a long felt need in applications where use of GMO's would be an acceptable benefit, such as the preparation of anthocyanins for use in medicinal compounds and compositions.

In addition to S. chilense, the fruit of several tomato species closely related to the cultivated tomato such as S. habrochaites, S. cheesmaniae, S. lycopersiciodes and S. pennelli v. puberelum contain significantly higher amounts of anthocyanins relative to cultivated tomatoes. Therefore introgression and expression of their respective AFT homologous genes into cultivated tomatoes would constitute a long awaited and novel advance. Also, introgression and expression of the AFT gene originating from S. chilense into tomato accessions that harbor flavonoid enhancing genes or alleles, other than AFT, can further increase flavonoid content.

As previously stated, certain increases in flavonoid content in the tomato fruit by transgenic genetic modification have been achieved, however consumer resistance to consuming GMO's is well known. There is therefore a demand in the marketplace for high flavonoid cultivars achieved through non-transgenic breeding techniques. Identification of the gene (or genes) that encode the AFT mutant phenotype in plant species in order to utilize the gene sequence as a DNA marker to expedite such breeding must therefore be regarded as a useful and novel attainment.

In light of the heightened interest in obtaining sources for edible antoxidants such as anthocyanins and flavonols, means and methods of producing tomato plants and other fruit plants with high anthocyanins, especially delphinidin, petunidin and malvidin and high flavonoid phenotypes, especially quercetin and kaempherol still remain a long felt need.

### SUMMARY OF THE INVENTION

It is one object of the present invention to disclose an AFT gene encoding a protein characterized by at least 80% identity with the amino acid sequence shown in FIG. 9 (LA1996 Seq.) having been genetically introgressed into cultivated plants or elite lines, conferring higher concentrations of flavonoids on the plants as compared with prior art cultivated plants that were not introgressed with the gene.

It is another object of the present invention to disclose the AFT gene, having been genetically introgressed into cultivated S. lycopersicum tomato plants or elite lines, conferring higher concentrations of flavonoids on the plants as compared with prior art cultivated S. lycopersicum plants that were not introgressed with the gene.

It is another object of the present invention to disclose the AFT gene originating from an S. chilense genotype having been genetically introgressed into cultivated S. lycopersicum tomato plants or elite lines, conferring higher concentrations of flavonoids on the plants as compared with prior art cultivated S. lycopersicum plants that were not introgressed with the gene.

It is another object of the present invention to disclose the AFT gene such that at least a portion of the flavonoids conferred by this gene are anthocyanins and/or flavonols.

It is another object of the present invention to disclose the AFT gene such that the gene originates from S. peruvianum.

It is another object of the present invention to disclose the AFT gene such that the gene is selected from a group consisting of S. habrochaites, S. cheesmaniae, S. lycopersiciodes, S. peruvianum and S. pennelli v. puberelum.

It is another object of the present invention to disclose the AFT S. chilense genotype introgressively-derived tomato plant, such that the plant is characterized by high concentrations of flavonoids as compared with prior art cultivated S. lycopersicum tomato plants that were not introgressed with said genotype.

It is still another object of the present invention to disclose the tomato plant such that the plant is characterized by high concentrations of anthocyanins and/or flavonols as compared with prior art cultivated S. lycopersicum tomato plants that were not introgressed with said genotype.

It is still another object of the present invention to disclose the tomato plant such that the AFT genotype is introgressed from S. peruvianum.

It is also an object of present invention to disclose the tomato plant such that the AFT genotype is introgressed from a group consisting of S. habrochaites, S. cheesmaniae S. lycopersiciodes, S. peruvianum and S. pennelli v. puberelum.

It is another object of the present invention to disclose the tomato plant obtained introgressively by a method comprising:
(i) crossing between hp-1/hp-1 accessions line tomatoes, especially S. lycopersicum, and tomatoes containing AFT gene from S. chilense;
(ii) selfing Ft plants resulting from said cross;
(iii) generating an F₂ population segregating for both the hp-1 mutation and the AFT allele;
(iv) selecting F₂ plants homozygous for the hp-1 mutation and the AFT locus from S. chilense; (v) selfing said F₂ plants to generate an F₃ population and further populations (F₄, F₅ and so forth) so as to obtain a pure-bred parental line characterized by high levels of flavonoids in a more than additive manner as compared with prior art cultivated S. lycopersicum tomato plants and/or initial parental lines; and,
(vi) using this parental line in crosses with other similar or different parental lines to obtain commercial F₁ hybrids.

It is a yet further object of the present invention to disclose the tomato plant such that the flavonoids include anthocyanins and flavonols, especially delphinidin, petunidin, malvidin, quercetin, kaempherol and naringenin.

It is another object of the present invention to disclose the tomato plant obtained introgressively by a method of crossing such that at least one parental is a high pigment accession line especially S. lycopersicum, the high pigment allele selected from a group characterized by one or more homozygotic alleles defined as hp-1, hp-1^{w}, hp-2, hp-2^{j}, hp-2^{dg}, such tomato plants crossed with plants containing AFT gene from S. chilense.

It is another object of the present invention to disclose the tomato plant, obtained introgressively by a method of crossing such that at least one parental is a high pigment accession line especially S. lycopersicum, the high pigment allele being selected from a group characterized by one or more homozygotic alleles at the UV-DAMAGED DNA BINDING PROTEIN 1 (DDB1) or DEETIOLATED1 (DET1) genes, such as: hp-1, hp-1^{w}, hp-2, hp-2^{j}, hp-2^{dg}, such tomato plants crossed with plants containing AFT gene from S. chilense.

It is another object of the present invention to disclose the tomato plant, obtained introgressively by a method of crossing such that least one parental is a high pigment accession line especially S. lycopersicum, the high pigment allele selected from a group characterized by one or more homozygotic alleles at photomorphogenic genes isophenotypic to hp-1, hp-1^{w}, hp-2, hp-2^{j}, hp-2^{dg}, the mutant plants being defective at the UV-DAMAGED DNA BINDING PROTEIN 1 (DDB1) or DEETIOLATED1 (DET1) genes, such tomato plants crossed with plants containing AFT gene from S. chilense.

It is another object of the present invention to disclose the tomato plant additionally comprising a step of selecting an F₂ plant homozygous at the AFT locus originating from S. chilense by means of a DNA marker.

It is another object of the present invention to disclose the tomato plant obtained as defined above, such that the AFT genotype originates from S. peruvianum.

It is another object of the present invention to disclose the tomato plant obtained as defined above, such that the AFT genotype is selected from a group consisting of S. habrochaites, S. cheesmaniae, S. lycopersiciodes, S. peruvianum and S. pennelli v. puberelum.

It is a further object of the present invention to disclose the tomato plant obtained as defined above, such that the DNA marker originates from S. peruvianum.

It is a further object of the present invention to disclose the tomato plant obtained as defined above, such that the DNA marker originates from a group consisting of S. habrochaites, S. cheesmaniae, S. lycopersiciodes, S. peruvianum and S. pennelli v. puberelum.

It is a further object of the present invention to disclose the tomato plant such that the flavonoid is selected from any member of a group consisting of the flavonoid aglycones, flavonoid O-glycosides, flavonoid C-glycosides, flavonoids with hydroxyland/or methoxy substitutions, C-methylflavonoids, methylenedioxy flavonoids chalcones, aurones, dihydrochalcones, flavanones, dihydroflavanols, anthoclors, proanthocyanidins, condensed proanthocyanidins, leucoanthocyanidins, flavan-3,4-ols, flavan-3-ols, glycosylflavonoids, biflavonoids, triflavonoids, isoflavoneoids, isoflavones, isoflavanones, rotenonoids, pterocarpans, isoflavans, quinone derivatives, 3-Aryl-4-hydroxycoumarins, 3-arylcoumarin, isoflav-3-enes, coumestans, α-methyldeoxybenzoins, 2-arylbenzofurans, isoflavanol, and coumaronochromone.

It is a further object of the present invention to disclose the tomato plant as defined above such that the anthocyanin is selected from a group consisting of delphidin, petundin or malvidin.

It is a further object of the present invention to disclose the tomato plant as defined above, such that the flavonoid is selected from a group consisting of quercetin and kaempherol.

It is a further object of the present invention to disclose the tomato plant as defined above, such that the flavonoid is selected from a group consisting of 4,2,4,6-tetrahydroxychalcone, naringenin, kaempherol, dihydroxy kaempherol, myrecetin, quercetin, dihydroquercetin, dihydromyrecetin, leucopelargonidin, leucocyanidin, leucodelphinidin, pelargonidin-3-glucoside, cyanidin-3-glucoside and delphinidin-3-glucoside.

It is a further object of the present invention to disclose the tomato plant as defined above such that the flavonoid is selected from a (i) group consisting of secondary plant metabolites derived from the 2-phenylchromone (2-phenyl-1,4-benzopyrone) structure; (ii) isoflavonoids, wherein said metabolites are derived from the 3-phenylchromone (3-phenyl-1,4-benzopyrone) structure; and, (iii) neoflavonoids wherein said metabolites are derived from the 4-phenylcoumarine (4-phenyl-1,2-benzopyrone) structure.

It is a further object of the present invention to disclose a DNA sequence which encodes for a protein characterized by at least 80% homology with the amino acid sequence shown in FIG. 9 (LA1996 Seq.) providing high flavonoid concentrations in tomato plants as compared with prior art cultivated S. lycopersicum tomato plants.

It is a further object of the present invention to disclose a DNA sequence according to claim 24, characterized by at least 80% homology with the nucleic acid sequence shown in the lower row of FIG. 2 (LA1996 Seq.) from residue 1 to residue 1008, providing high flavonoid concentrations in tomato plants as compared with prior art cultivated S. lycopersicum tomato plants.

It is a further object of the present invention to disclose a DNA sequence conferring accumulation or expression of metabolites of the flavonoid pathway, especially anthocyanin or flavonols, in plants, plant parts or seeds thereof.

It is a further object of the present invention to disclose a DNA sequence conferring accumulation or expression of metabolites of the flavonoid pathway, especially anthocyanin or flavonols, in tomato plants, especially S. lycopersicum, tomato plant parts or seeds thereof.

It is a further object of the present invention to disclose a DNA sequence found useful in screening germplasm, seeds, seedlings, cali, plants or plant parts for introgression of the AFT genotype in cultivated tomato accessions such that the DNA is characterized by at least 80% homology with the nucleic acid sequence shown in the lower row of FIG. 2 (LA1996 Seq.) from residue 1 to residue 1008.

It is a further object of the present invention to disclose a transgenic plant expressing metabolites of the flavonoid pathway, especially anthocyanin or flavonols, in plants, plant parts or seeds thereof, the plant comprising DNA with at least 80% homology with the nucleic acid sequence shown in the lower row of FIG. 2 (LA1996 Seq.) from residue 1 to residue 1008; the DNA recombined into a plurality of one or more transformation and/or expression vectors, useful for transformation and/or expression in plants.

It is within the scope of the present invention to disclose a method of obtaining an AFT gene encoding a protein characterized by at least 80% identity with the amino acid sequence shown in FIG. 9 (LA1996 Seq.), the gene having been genetically introgressed into cultivated plants or elite lines, conferring higher concentrations of flavonoids on the plants as compared with prior art cultivated plants that were not introgressed with the AFT gene.

It is within the scope of the present invention to disclose a method of obtaining AFT S. chilense genotype introgressively-derived tomato plants, characterized by high concentrations of anthocyanins and/or flavonoids as compared with prior art cultivated S. lycopersicum tomato plants; said method comprising of
(i) crossing between hp-1/hp-1 accessions line tomatoes, especially S. lycopersicum, and tomatoes containing AFT gene from S. chilense;
(ii) selfing F₁ plants resulting from the cross;
(iii) generating an F₂ population segregating for both the hp-1 mutation and the AFT allele;
(iv) selecting F₂ plants homozygous for the hp-1 mutation and the AFT locus from S. chilense;
(v) selfing said F₂ plants to generate an F₃ population so as to obtain a pure-bred parental line characterized by high levels of flavonoids in a more than additive manner as compared with prior art cultivated S. lycopersicum tomato plants and/or initial parental lines; and,
(vi) using this parental line in crosses with other similar or different parental lines to obtain commercial F₁ hybrids.

It is within the scope of the present invention to disclose a method of obtaining AFT S. chilense genotype introgressively-derived tomato plants, characterized by high concentrations of anthocyanins and/or flavonoids as compared with prior art cultivated S. lycopersicum tomato plants; the method comprising of:
(i) crossing between hp-1/hp-1 accessions line tomatoes, especially S. lycopersicum, and tomatoes containing AFT gene from S. chilense;
(ii) selfing F₁ plants resulting from the cross;
(iii) generating an F₂ population segregating for both the hp-1 mutation and the AFT allele;
(iv) selecting F₂ plants homozygous for the hp-1 mutation and the AFT locus from S. chilense;
(v) selfing said F₂ plants to generate an F₃ and further populations (F₄, F₅ and so forth) so as to obtain a pure-bred parental line characterized by high levels of anthocyanins and flavonols especially delphinidin, petunidin, malvidin, quercetin and kaempherol, in a more than additive manner as compared with prior art cultivated S. lycopersicum tomato plants and/or initial parental lines; and,
(vi) using this parental line in crosses with other similar or different parental lines to obtain commercial F₁ hybrids.

It is within the scope of the present invention to disclose a method of obtaining introgressed plants by crossing as above such that at least one parental is a high pigment accession line especially S. lycopersicum, and so that high pigment alleles are selected from a group characterized by one or more homozygotic alleles defined as hp-1^{w}, hp-2, hp-2^{j}, hp-2^{dg}, such tomato plants then being crossed with plants containing AFT gene from S. chilense.

It is within the scope of the present invention to disclose a method of obtaining introgressed plants by crossing wherein at least one parental is a high pigment accession line especially S. lycopersicum, such that the high pigment alleles are selected from a group characterized by one or more homozygotic alleles at the UV-DAMAGED DNA BINDING PROTEIN 1 (DDB1) or DEETIOLATED1 (DET1) genes, such as: hp-1, hp-1^{w}, hp-2, hp-2^{j}, hp-2^{dg}, the tomato plants then being crossed with plants containing AFT gene from S. chilense.

It is well within the scope of the present invention to disclose a method of obtaining introgressed plants by crossing such that at least one parental is a high pigment accession line especially S. lycopersicum, and that the high pigment alleles are selected from a group characterized by one or more homozygotic alleles at photomorphogenic genes isophenotypic to hp-1, hp-1^{w}, hp-2, hp-2^{j}, hp-2^{dg} mutant plants defective at the UV-DAMAGED DNA BINDING PROTEIN 1 (DDB1) or DEETIOLATED1 (DET1) genes, such tomato plants then being crossed with plants containing AFT gene from S. chilense.

It is still within the scope of the present invention to disclose a method of obtaining introgressed plants as described above, additionally comprising of selecting by means of a DNA marker an F₂ plant homozygous for the HP-1 and AFT loci.

It is still within the scope of the present invention to disclose a method of obtaining introgressed plants as described above such that the anthocyanin is selected from a group consisting of delphidin, petundin and malvidin.

It is still within the scope of the present invention to disclose a method of obtaining introgressed plants as described above so that the flavonoid is selected from a group consisting of quercetin and kaempherol.

It is still within the scope of the present invention to disclose a method for obtaining a tomato plant with high flavonoids as compared with prior art cultivated S. lycopersicum tomato plants, such that the flavonoid is selected from a group consisting of 4,2,4,6-tetra hydroxychalcone, naringenin, kaempherol, dihydroxy kaempherol, myrecetin, quercetin, dihydroquercetin, dihydromyrecetin, leucopelargonidin, leucocyanidin, leucodelphinidin, pelargonidin-3-glucoside, cyanidin-3-glucoside and delphinidin-3-glucoside.

It is still well within the scope of the present invention to disclose a method for obtaining a tomato plant such that the flavonoids are selected from a group consisting of secondary plant metabolites derived from (i) 2-phenylchromone (2-phenyl-1,4-benzopyrone) structure; (ii) isoflavonoids, wherein said metabolites are derived from the 3-phenylchromone (3-phenyl-1,4-benzopyrone) structure; and (iii), a neoflavonoids wherein said metabolites are derived from the 4-phenylcoumarine (4-phenyl-1,2-benzopyrone) structure.

It is still well within the scope of the present invention to disclose a method for obtaining a tomato such that the flavonoids are selected from any member of a group consisting of flavonoid aglycones, flavonoid O-glycosides, flavonoid C-glycosides, flavonoids with hydroxyl and/or methoxy substitutions, C-methylflavonoids, methylenedioxyflavonoids chalcones, aurones, dihydrochalcones flavanones, dihydroflavanols, anthoclors, proanthocyanidins, condensed proanthocyanidins, leucoanthocyanidins, flavan-3,4-ols, flavan-3-ols, glycosylflavonoids, biflavonoids, triflavonoids, isoflavoneoids, isoflavones, isoflavanones, rotenonoids, pterocarpans, isoflavans, quinonederivatives, 3-Aryl-4 hydroxycoumarins, 3-arylcoumarin, isoflav-3-enes, coumestans, methyldeoxybenzoins, 2-arylbenzofurans, isoflavanol, and coumaronochromone.

It is still well within the scope of the present invention to disclose a method for obtaining DNA which encodes for a protein comprising at least 80% identity with an amino acid sequence shown in shown in FIG. 9 (LA 1996 seq.); the method comprised of identifying and optionally verifying the encoded amino acid sequence, the sequence being of a protein naturally occurring in S. chilense responsible for the AFT phenotype and enhanced flavonoid concentration.

It is still well within the scope of the present invention to disclose a method useful for obtaining nucleic acid characterized by at least 80% homology with the nucleic acid sequence shown in the lower row of FIG. 2 (LA1996 seq.) from residue 1 to residue 1008, the method comprising identifying and optionally verifying the nucleic acid sequence as encoding a protein naturally occurring in S. chilense responsible at least in part for the AFT phenotype.

Moreover, it is still well within the scope of the present invention to disclose a method for obtaining tomato plants high in flavonoids as compared with prior art cultivated S. lycopersicum tomato plants; facilitated by screening germ plasm, seeds, seedlings, cali or plants for introgression of the AFT S. chilense genotype into cultivated tomato accessions; the method comprising of:
(i) obtaining nucleic acid at least 80% homologous with the nucleic acid sequence shown in the lower row of FIG. 2 (LA1996 Seq.) from residue 1 to residue 1008;
(ii) preparing PCR primers as defined in Table 1;
(iii) amplifying DNA of (i); and,
(iv) probing target tissue therewith.

Furthermore, it is well within the scope of the present invention to disclose transgenic method for accumulating or expressing metabolites of the flavonoid pathway, especially anthocyanin or flavonols, in plants, plant parts or seeds thereof, the method comprising of:
(i) obtaining DNA at least 80% homologous with the nucleic acid sequence shown in the lower row of FIG. 2 (LA1996 Seq.) from residue 1 to residue 1008; and,
(ii) combining said DNA into a plurality of one or more transformation and/or expression vectors, useful for transformation and/or expression in plants.

Lastly, it is well within the scope of the present invention to disclose a transgenic method for accumulating or expressing metabolites of the flavonoid pathway, especially useful for tomato plants, tomato plant parts or seeds thereof.

### BRIEF DESCRIPTION OF THE FIGURES

In order to understand the invention and to see how it may be implemented in practice, preferred embodiments will now be described, by way of non limiting examples only, with reference to the accompanying drawings in which

FIG. 1 schematically presents the anthocyanin and flavonol biosynthetic pathway (adopted from Holton and Cornish, (1995) Plant Cell 7:1071-1083);

FIG. 2 presents a schematic nucleotide sequence comparison of the ANT1 gene between cv. Ailsa Craig (upper rows) and LA 1996 (lower rows) [start and stop codons are underlined in both sequences, and intronic regions are highlighted in yellow];

FIG. 3 schematically presents an amino-acid comparison of the ANT1 protein between cv. Ailsa Craig (upper rows) and LA 1996 (lower rows) [Amino acids that differ between the two lines are highlighted in yellow];

FIG. 4 presents photographic representations of co-dominant polymorphisms between the ANT1 alleles originating from S. lycopersicum (ANT1^{L}) and from S. chilense (ANT1^{C});

FIG. 5 presents a visual display of the association between the ANT1 gene and that trait of anthocyanin accumulation in F₂ population segregating for ANT1 and hp-1 (each fruit was harvested from an individual plant of the respective genotype);

FIG. 6 presents photographic and schematic representations illustrating restriction enzyme mapping of ANT1 to the tomato genome (map of the tomato chromosome 10 was adopted from http://tgrc.ucdavis.edu/pennellii-ILs.pdf);

FIG. 7 presents a photographic comparison between tobacco regenerants transformed with the ANT1 gene originating from S. chilense (ANT1^{C}) and S. lycopersicum (ANT1^{L}) under the control of cauliflower mosaic virus 35S constitutive promoter;

FIG. 8 presents a photographic comparison between tomato (cv. Moneymaker) regenerants transformed with the ANT1 gene originating from S. chilense (ANT1^{C}) and S. lycopersicum (ANT1^{L}) under the control of cauliflower mosaic virus 35S constitutive promoter;

FIG. 9 presents a schematic amino acid alignment of the ANT1 gene cloned from tomato accessions and pepper (Accessions that do not accumulate fruit anthocyanins: LA1589 is S. pimpinellifolium, LA2838A is S. lycopersicum; Accessions that do accumulate fruit anthocyanins: PI128650 is S. peruvianum, hp-799 is a selection line originating from a cross between an unknown S. peruvianum and S. lycopersicum, LA1996 is an AFT genotype originating from S. chilense, CAE75745 is an anthocyanin accumulating pepper);

FIG. 10 presents tomato fruits harvested from LA1996 plant (ANT1^{C}/ANT1^{C}+/+) and F₃ plants homozygous for both the hp-1 mutation and the ANT1^{C} allele (ANT1^{C}/ANT1^{C} hp-1/hp-1) according to one embodiment of the present invention; and,

FIG. 11 presents a tomato plant and fruits of an accession that is homozygous for the hp-1 mutation and the ANT1 allele originating from S. peruvianum (ANT1^{P}/ANT1^{P} hp-1/hp-1), according to another embodiment of the present invention.

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications, however will remain apparent to those skilled in the art since the generic principles of the present invention have been defined specifically to provide means and methods of producing tomato plants and other fruit plants with high anthocyanins, especially delphinidin, petunidin and malvidin and high flavonoid phenotypes, especially quercetin and kaempherol.

The term “hp-1” refers hereafter to a mutation of the HIGH PIGMENT-1 gene which, when introduced into commercial tomato cultivars enriches their fruits with higher levels of carotenoids, flavenoids and vitamins C and E. The mutation hp-1 belongs to an isophenotypic group of mutations that include hp-1^{w}, hp-2, hp-2^{j}, hp-2^{dg} that map to the tomato UV-DAMAGED DNA BINDING PROTEIN 1 (DDB1) and DEETIOLATED1 (DET1) genes.

The term “ANTHOCYANIN FRUIT (AFT)” refers hereinafter to a specific single gene, conferring high levels of anthocyanins and other flavonoid metabolites on cultivated tomatoes such as S. lycopersicum, when introgressed from S. chilense.

The term “ANT1^{L}” refers hereinafter to a specific single gene of S. lycopersicum responsible for anthocyanin and flavonoid accumulation.

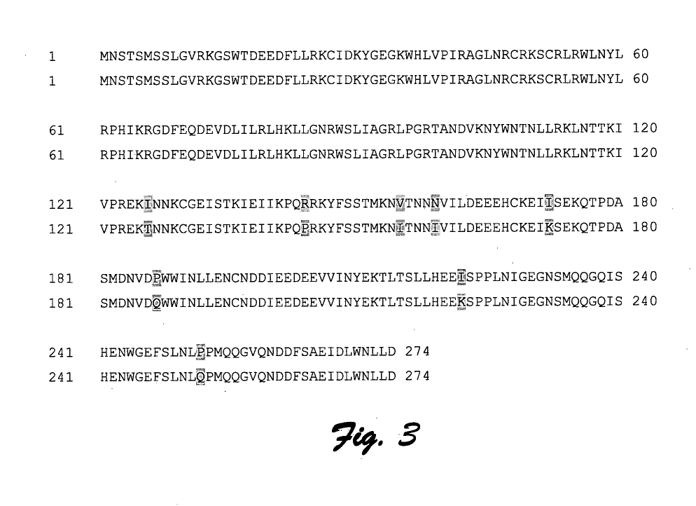

The term “ANT1^{C}” refers hereinafter to a specific single gene of S. chilense responsible for anthocyanin and flavonoid accumulation. The polypeptide encoded by ANT1 differs from ANT1^{L} by 8 amino acid changes (FIG. 3).

The term “introgression” or “introgressively derived” refers hereinafter to the plant breeding technique whereby a gene is moved from one species to the gene pool of another species or accession by crossing and backcrossing, that is accompanied by selection of desirable genotypes and phenotypes. A DNA marker can facilitate the choice of a desirable genotype, and thereby expedite breeding.

The term “transformation” refers hereinafter to any method of introducing a heterologous plant DNA sequence, possibly incorporated within any type of DNA vector system or construct, permanently into the target host plant genome or cytoplasm, introduction of said plant DNA construct being accomplished by a variety of techniques known in the art.

The term “plant” or “plant part’ refers hereinafter to any plant, plant organ or tissue including without limitation, fruits, seeds, embryos, meristematic regions, callus tissue, flowers, leaves, roots, shoots, gametophytes, sporophytes pollen, and microspores. Plant cells can be obtained from any plant organ or tissue and cultures prepared therefrom. The class of plants which can be used in the methods of the present invention is generally as broad as the class of higher plants amenable to transformation techniques, including both monocotelydenous and dicotelydenous plants.

The term “flavonoid” refers hereinafter to any plant secondary metabolites, defined according to the IUPAC nomenclature as (i) flavonoids, especially wherein the metabolite is derived from the 2-phenylchromone (2-phenyl-1,4-benzopyrone) structure; (ii) isoflavonoids, wherein the metabolite is derived from the 3-phenylchromone (3-phenyl-1,4-benzopyrone) structure; and (iii) neoflavonoids, wherein the metabolite is derived from the 4-phenylcoumarine (4-phenyl-1,2-benzopyrone) structure. Equally the term may refer to any of the flavonoid aglycones, flavonoid O-glycosides, flavonoid C-glycosides, flavonoids with hydroxyland/or methoxy substitutions, C-methylflavonoids, methylenedioxyflavonoids, chalcones, aurones, dihydrochalcones, flavanones, dihydroflavanols, anthoclors, proanthocyanidins, condensed proanthocyanidins, leucoanthocyanidins, flavan-3,4-ols, flavan-3-ols, glycosylflavonoids, biflavonoids, triflavonoids, isoflavoneoids, isoflavones, isoflavanones, rotenonoids, pterocarpans, isoflavans, quinone derivatives, 3-aryl-4-hydroxycoumarins, 3-arylcoumarin, isoflav-3-enes, coumestans, α-methyldeoxybenzoins, 2-arylbenzofurans, isoflavanol, and coumaronochromone.

The term “flavonol” refers hereinafter to any flavonoid possessing the 3-hydroxy-2-phenyl-4H-1-benzopyran-4-one backbone as defined by IUPAC. Their diversity stems from the different positions of the phenolic —OH groups, exemplified in a non-limiting manner by quercetin (3,5,7,3′,4′-pentahydroxy-2-phenyl-4H-1-benzopyran-4-one), kaempferol (3,5,7,4′-tetrahydroxy-2-phenyl-4H-1-benzopyran-4-one) and myricetin (3,5,7,3′,4′,5′-hexahydroxy-2-phenyl-4′-1-benzopyran-4-one).

The term “anthocyanidin”, refers hereinafter to any flavenoid possessing an oxygen-containing heterocycle pyran fused to a benzene ring wherein the pyran ring is connected to a phenyl group at the 2-position, which can carry different substituents.

The term “anthocyanin’, refers hereinafter to an anthocyanidin possessing any sugar moiety.

The term “cv.” refers to commercially or non-commercially available cultivars.

The term “elite” refers hereinafter to any commercial plant hybrid, especially tomato.

### Plant Material, Crosses and Growth Conditions

The following plant materials were obtained: Accession LA1996 containing S. chilense AFT. Moneymaker (red-fruited open-pollinated fresh-market type tomato). Ailsa Craig (red-fruited open-pollinated tomato and nearly isogenic and homozygous for the hp-1 mutation). VF36 (LA0490) (red-fruited open-pollinated cultivars). Rutgers (LA1090) (red-fruited open-pollinated cultivars). LA 1589 (red fruited S. pimpinellifolium accession). PI128650 purple fruited S. peruvianum accession.

A cross was made between cv., Moneymaker and Ailsa Craig hp-1/hp-1 as a maternal line and LA1996 as a paternal line. F₁ plants resulting from this cross were allowed to self-pollinate to generate an F₂ population segregating for both the hp-1 mutation and the AFT allele. A plant homozygous for the hp-1 mutation and heterozygous at the AFT locus, based on a DNA marker disclosed herein, was selected from the above F₂ population and allowed to self-pollinate in order to generate an F₃ population segregating for the AFT in hp-1/hp-1 background.

Plants were planted and grown at two locations in central Israel- at the Volcani Center and on the premises of Zeraim Gedera Seed Company (IL). During the summer season-plants were grown in the open-field and/or in a screen-house, and during the winter seasons in a controlled heated greenhouse; minimal temperature 15° C. Transplanting for the summer seasons was carried out during the first week of May, whereas in the winter seasons, transplanting was carried out during the first week of November.

### Genomic DNA Extraction

Genomic DNA was extracted from individual plants according to Fulton et al., (1995) Plant Mol Biol Rep 13: 207-209).

### Design of Polymerase Chain Reaction (PCR) Primers

Sequence analysis and locus-specific primer design were carried out with the DNAMAN sequence analysis software v 4.1 (Lynnon BioSoft, Quebec).

### Pyrosequencing Genotyping

A pyrosequencing system was used to genotype for the hp-1 mutation. This pyrosequencing genotyping system is based on a single nucleotide polymorphism, discovered in the gene that encodes the hp-1 mutant phenotype, between the hp-1/hp-1 mutant plants and their nearly isogenic counterparts. The genotyping procedure used was as described by Lieberman et al., (2004) Theor Appl Genet. 108, 1574-1581).

### Genotyping of ANT1 and Other Structural Genes of the Flavonoid Biosynthetic Pathway

Genotyping was carried out for the purpose of linkage analysis, and/or polymorphism determination using PCR followed by restriction endonuclease digestion. The primers used in these PCR amplifications are presented in Table 1. PCR amplification products were visualized by electrophoresis in 1.0% agarose gels stained with ethidium bromide.

TABLE 1
Forward (F) and reverse (R) primers used for
genotyping the ANT1 and other structural genes of
the flavonoid biosynthetic pathway.

| Gene | Primers |
|---|---|
| ANT1 | F = 5′-GGAAGGACAGCTAACGATGTG-3′ |
| | R = 5′-GTTGCATGGGTGGTAAATTAAG-3′ |
| CHS1 | F = 5′-TGAGATCACTGCAGTTACGTTC-3′ |
| | R = 5′-TAAGCCCAGCCCACTAAGC-3′ |
| CHS2 | F = 5′-TCTGCAGCCCAAACTCTAC-3′ |
| | R = 5′-TATGGAGCACAACAGTCTCAACA-3′ |
| DFR | F = 5′-GATAAGGACTTGGCCCTAGTG-3′ |
| | R = 5′-GATACGCGAGAGCCTTCAG-3′ |
| F3H | F = 5′-CCAATCAAAGACGCATTAGCAG-3′ |
| | R = 5′-TCACAAGGAAGGCCAAGATAAAG-3′ |

### Real-time PCR Analysis

RNA was extracted from fruit peels of mature-green tomato fruits. Three squares of 1 mm² from each genotype (LA1996, the normal red-fruited genotypes Moneymaker, VF36, and Rutgers, and the F₁ hybrids between LA1996 and Moneymaker) were analyzed in each experiment. The RNA extraction was carried out using TRIzol reagent system (Invitrogen Corp., Carlsbad, Calif.). Possible genomic DNA contaminants were digested with TURBO DNA-free (Ambion Inc., Austin, Tex.) and the total RNA was then used as the template for cDNA synthesis using the iScript cDNA synthesis kit (Bio-Rad Laboratories, Hercules, Calif.). The purity of the total RNA and the quality of the synthesized cDNA were verified by PCR using any of the primers mentioned herein below.

The real-time PCR analysis was performed using the SYBER GREEN PCR Master Mix (Applied Biosystems, Foster City, Calif.). The PCR reaction was carried out using initial incubation at 95° C. for 10 min, followed by 40 cycles of denaturation at 95° C. for 15 sec, annealing at 61° C. for 30 sec, and polymerization at 72° C. for 30 sec.

18S ribosomal RNA was used as reference gene throughout this study and the primers designed for it, as well as for the other genes analyzed—CHALCONE SYNTHASE1 (CHS1), CHALCONE SYNTHASE2 (CHS2), CHALCONE ISOMERASE (CHI), DIHYDROFLAVONOL REDUCTASE (DFR), FLAVANONE-3-HYDROXYLASE (F3H), and ANTHOCYANIN1 (ANT1), are presented in Table 2. Samples were analyzed usually in duplicates, using the GeneAmp 5700 Sequence Detection System and data was collected and analyzed with the GeneAmp 5700 SDS software (Applied Biosystems). The relative abundance of the examined genes transcripts was calculated by the formula: 2^{(CT}^{_{—}}^{examine gene-CT}^{_{—}}^{reference gene)}, where C_{T} represents the fractional cycle number at which the fluorescence crosses a fixed threshold (usually set on 0.1).

TABLE 2
Forward (F) and reverse (R) primers used
for real-time PCR analysis.

| Gene | Primer sequence |
|---|---|
| CHS1 | F = 5′-GCCTCTACAAAAGAAGGCCTAG-3′ |
| | R = 5′-TAAGCCCAGCCCACTAAGC-3′ |
| CHS2 | F = 5′-CATCCAAAGAAGGGCTTAGTACC-3′ |
| | R = 5′-TATGGAGCACAACAGTCTCAACA-3′ |
| CHI | F = 5′-TTTCAAGGCTTCCAGGATATG-3′ |
| | R = 5′-ATGTCCCGAACTTCTCCTTG-3′ |
| DFR | F = 5′-TTCAAGTGGCAAGGAGAATG-3′ |
| | R = 5′-AGAACATGTTGGTGAGGTAGCTC-3′ |
| F3H | F = 5′-GTGAATCCTAGCCTTGACAGTG-3′ |
| | R = 5′-GCTTTACCAGAAGGCCTCTTAC-3′ |
| ANT1 | F = 5′-GACGCAAGTATTTCTCAAGCAC-3′ |
| | R = 5′-TCCACCATGGATCTACGTTG-3′ |
| 18S ribosomal | F = 5′-GCGACGCATCATTCAAATTTC-3′ |
| RNA | R = 5′-TCCGGAATCGAACCCTAATTC-3′ |

### Anthocyanin and Flavonol Extraction and Quantification

Samples of fresh tomato skin (0.1 to 0.3 g) were ground in liquid nitrogen and the pigments were extracted in the dark with 2 ml of cold methanol:water:acetic acid (11:5:1); (Markham and KR Ofman, (1993) Phytochemistry 34: 679-685.).

Extracts were spun for 10 min at 20,800 g (14,000 rpm), leaving the anthocyanins in the supernatant. Further purifications were with 2/3 volumes of hexane. Samples were then concentrated to 0.5 ml, hydrolyzed by boiling with equal volume of methanol and in 2 N HCl for 1 h and passed through a 0.45 μm polyvinylidene difluoride filter (Nalgene).

Flavonoid compositions were determined using a HPLC (Shimatzu, JP) equipped with a LC-10AT pump, a SCL-10A controller and a SPD-M10AVP photodiode-array detector. Extracts were loaded onto a RP-18 column (Vydac 201TP54) and separated at 27° C. with the following solutions: (A) H₂O, pH 2.3 and (B) H₂O:MeCN:HOAc (107:50:40), pH 2.3. Solutions were applied as a linear gradient from a ratio of 4:1 (A:B) to 3:7 over 45 min, and held at a ratio of 3:7 for an additional 10 min at a flow rate of 0.5 ml/min, flavonoids were identified by comparing both the retention time and the absorption spectrum at 250-650 nm with those of standard purified flavonoids (Apin chemicals, Polyphenols, Sigma).

### Mapping the ANT1 and AFT Genes to the Tomato Genome

The AFT gene, found in the course of this study to be highly associated to the ANT1 gene, was mapped to the tomato genome by means of S. pennellii introgression lines (Eshed et al., (1992) Theor Appl Genet. 83: 1027-1034), as was earlier demonstrated (Levin et al. (2000) Theor Appl Genet. 100: 256-262,).

DNA extracted from individual plants of each of the introgression lines, including their original parental lines M82 and S. pennellii, were used as templates in PCR reactions. Reactions were carried out in an automated thermocycler (MJ research, Waltham, Mass.). The DNA primers used for these reactions were (Mathews et al., 2003, Plant Cell 15: 1689-1703):

F: 5′-TCCCCCGGGATGAACAGTACATCTATG-3′
and
R: 5′-GGACTAGTTTAATCAAGTAGATTCCATAAGTCA-3′.

The PCR reaction was carried out using initial incubation at 94° C. for 3 min, followed by 35 cycles of denaturation at 94° C. for 30 sec, annealing at 60° C. for 30 sec, and polymerization at 72° C. for 60 sec. A final elongation step at 72° C. was carried out for 7 min following the completion of the above cycles. The PCR products obtained were visualized by electrophoresis 1 m 1.0% agarose gel which was stained with ethidium bromide. Restriction endonuclease digestion was not needed in order to obtain polymorphism between the parental lines: M-82 (LA3475) and S. pennellii (LA0716).

### Cloning of ANT1 Gene

Total RNA was extracted from 100 mg of leaf tissue of individual AFT mutant (LA1996) plants and a wild-type genotype (Ailsa Craig). The RNA extraction was carried out with the TRIzol reagent system (Invitrogen Corp., Carlsbad, Calif.). Possible genomic DNA contaminants were digested with TURBO DNA-free (Ambion Inc., Austin, Tex.) and the total RNA was then used as the template for cDNA synthesis using the iScript cDNA synthesis kit (Bio-Rad Laboratories, Hercules, Calif.). The cDNA prepared was used as a template in PCR reactions to amplify the ANT1 gene sequence (GenBank accession number AY348870), from both mutant and normal genetic accessions. The PCR was carried out with proof-reading Tag polymerase (Pwo DNA Polymerase, Roche Diagnostics Corp., Indianapolis, Ind., USA) using specific primers for 5′ and 3′ ends of the gene:

F: 5′-ATGAACAGTACATCTATGTCTTCATTGG-3′;
and
R: 5′-GGACTAGTTTAATCAAGTAGATTCCATAAGTC-3′.

The resulting product was ligated into pCRII-TOPO vector (Invitrogen Corp., Carlsbad, Calif.) after TA cloning and verified by sequence analysis.

### Construction of Binary Vectors for Plant Transformation

The constructs for plant transformation bearing the mutant and the normal ANT1 under control of cauliflower mosaic virus (CaMV) 35S constitutive promoter, based on pMON10098 plasmid, were prepared. All of these constructs had NPTII selectable marker gene, also under the 35S promoter.

To prepare the constructs, pMON10098 plasmid was digested with EcoRI followed by treatment with Shrimp alkaline phosphatase (Roche Diagnostics Corp., Indianapolis, Ind., USA) and ligated with EcoRI-digested ANT1 gene (from the pCRII-TOPO vector). The clone containing pMON-35S-ANT1 in tandem was isolated and its sequence verified for both mutant and normal ANT1 clones.

### Transformation Protocol

Leaf cuttings of Nicotiana tobacum SR1 and cotyledon cuttings of Solanum lycopersicum cv Moneymaker were used for transformation of both of the above constructs.

Culture initiation was as follows: plant seeds were washed with soap (commercially available Palmolive) and water then placed in water and washed under running tap water for 1.5 hours. Seeds were then shaken in 96% v/v ethanol for 1 minute and placed for 15 minutes in 3% v/v sodium hypochlorite+0.01% v/v Tween-20, and for 30 minutes in 1.5% v/v Sodium-hypochlorite+0.01% Tween-20 with vigorous mixing. Lastly, the seeds were washed 3 times with sterile distilled water. Leaf and cotyledons cuttings were detached from the bases of stems of seedlings obtained from the above seeds, and were placed on MS media differing in their plant growth regulators content, as described below.

The above explants were placed on a basic media that are standard in our lab and contain MS salts (Murashige and Skoog, (1962) Physiol Plant 15(3): 473-497) supplemented with 30 sucrose or glucose and 8 g 1-1 Agar. Growth regulators supplements (in mg 1-1) were as follows: for tobacco 0.8 IAA and 2 Kinetin and 1 Zeatin and for tomato: 0.1 IAA and 1 Zeatin.

Cultures were maintained in a culture room at 23° C. under 16 h light (cool white fluorescent lamps giving 50 □mol m-2 s-1) regime. All explants were placed on the above media and were evaluated for their ability to regenerate shoots.

All vectors used throughout this study were inserted into the Agrobacterium tumefaciens strain EHA105. Leaf and cotyledon cuttings were incubated under sterile conditions with Agrobacterium in liquid MS medium supplemented with 200 μM acetosyringone for 20 minutes. After blotting the tissue with sterile filter paper the callus pieces were co-cultivated in MS medium with 100 μM acetosyringone in darkness at 22° C. Subsequently, the cuttings were washed and transferred to solid regeneration medium containing 50 mg l⁻¹ kanamycin for selection and 200 mg l⁻¹ cefotaxime and grown in a culture chamber.

### Statistical Analyses

Analyses of variance (ANOVA) were carried out with the JMP Statistical Discovery software (SAS Institute, Cary, N.C.). Alignment of nucleotide and amino-acid sequences was carried out using the CLUSTAL W method (Thompson et al., (1994) Nucleic Acids Res 22: 4673-4680) utilizing the Biology WorkBench at http://workbench.sdsc.ede/.

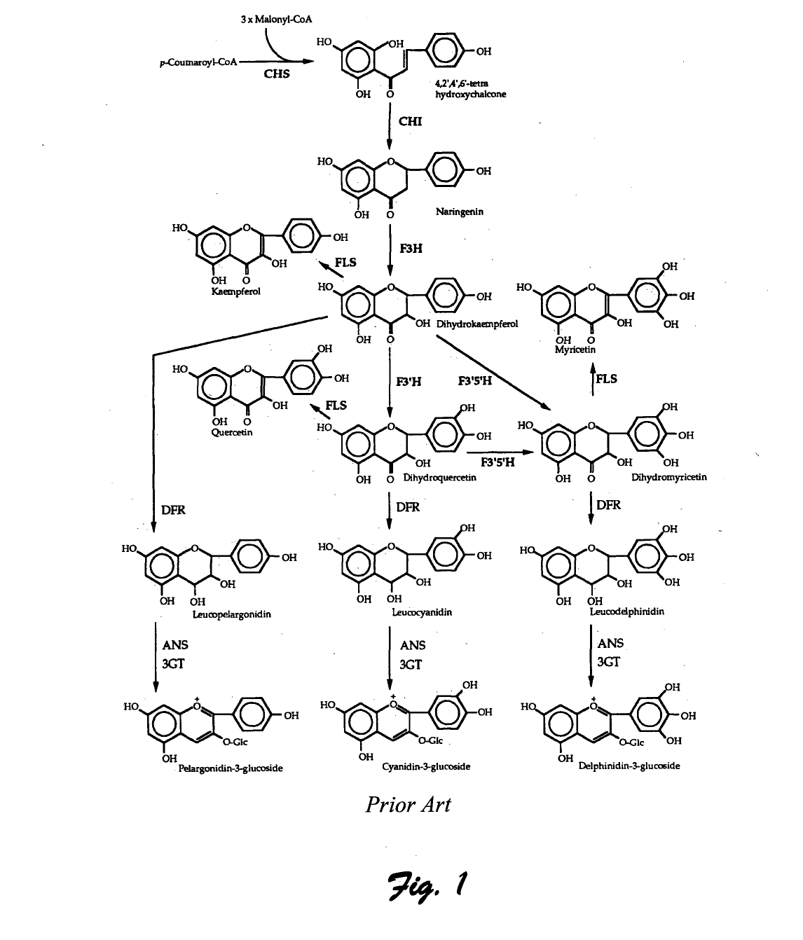

Reference is now made to FIG. 1, presenting a schematic illustration of anthocyanin and flavonol biosynthetic pathway according to prior art.

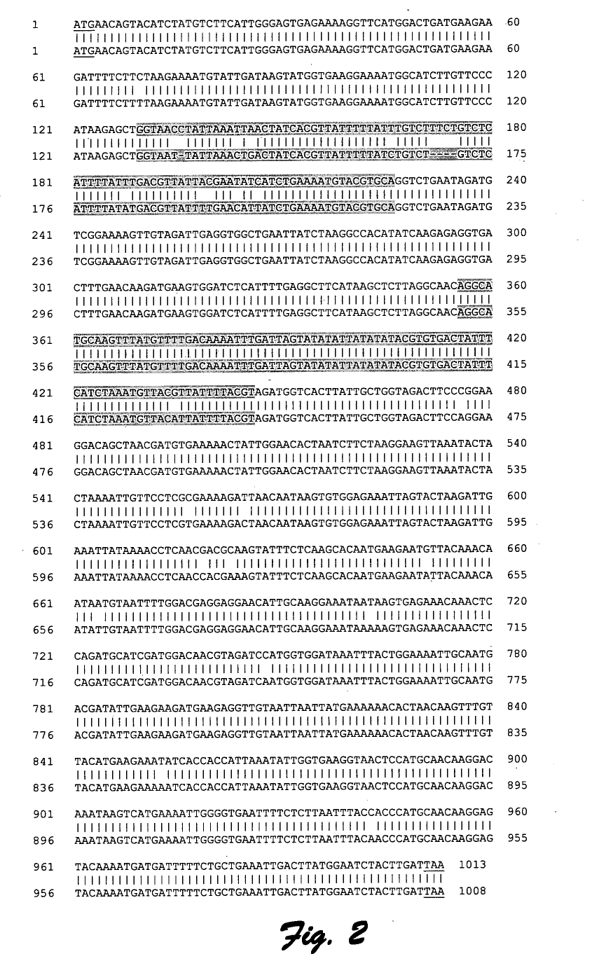

Reference is now made to FIG. 2, presenting a schematic nucleotide sequence comparison of the ANT1 gene between cv. Ailsa Craig (upper rows) and LA 1996 (lower rows) [start and stop codons are underlined in both sequences, and intronic regions are highlighted in yellow].

Reference is now made to FIG. 3, presenting a schematic amino-acid comparison of the ANT1 protein between cv. Ailsa Craig (upper rows) and LA 1996 (lower rows) [Amino acids that differ between the two lines are highlighted].

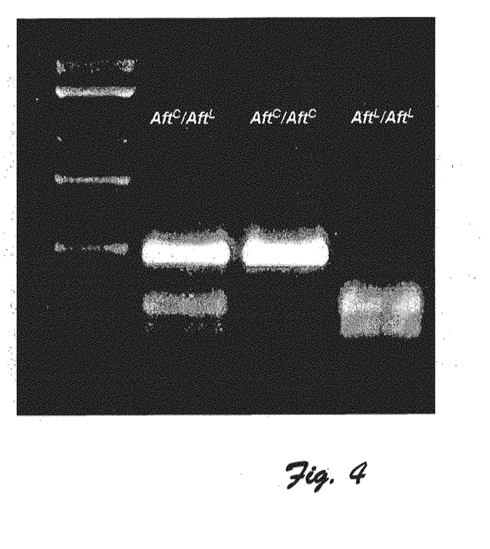

Reference is now made to FIG. 4, presenting a photographic illustration of co-dominant polymorphisms between the ANT1 alleles originating from S. lycopersicum (ANT1^{L}) and from S. chilense (ANT1^{C}).

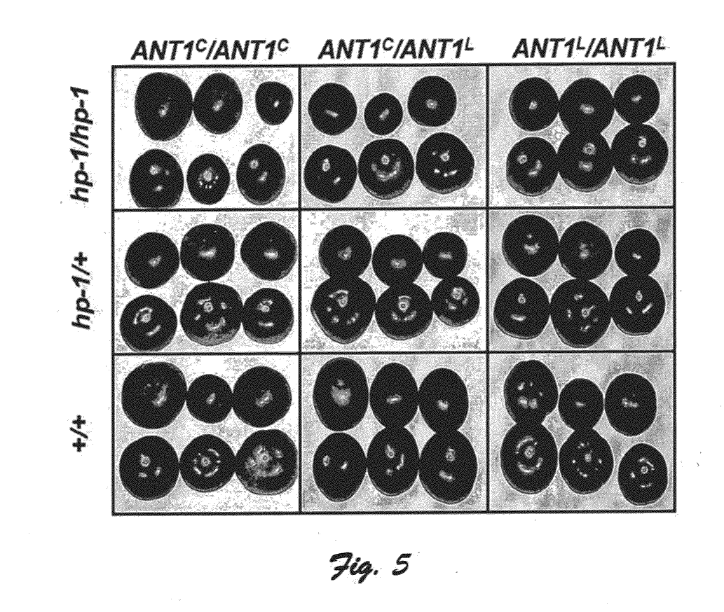

Reference is now made to FIG. 5, presenting a photographic illustration of the association between the ANT1 gene and that trait of anthocyanin accumulation in F₂ population segregating for ANT1 and hp-1 (each fruit was harvested from an individual plant of the respective genotype).

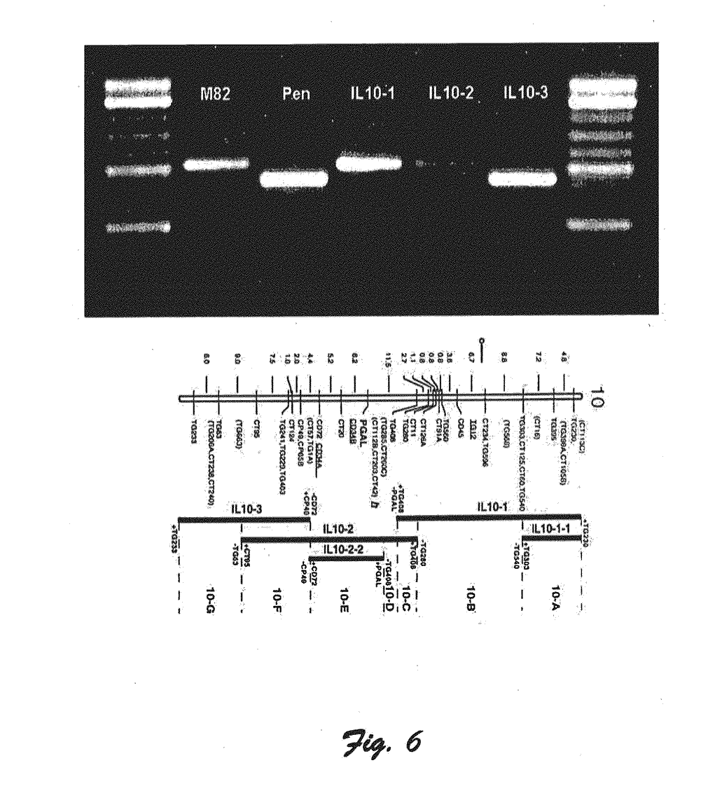

Reference is now made to FIG. 6, presenting a photographic and schematic mapping of ANT1 to the tomato genome on chromosome 10.

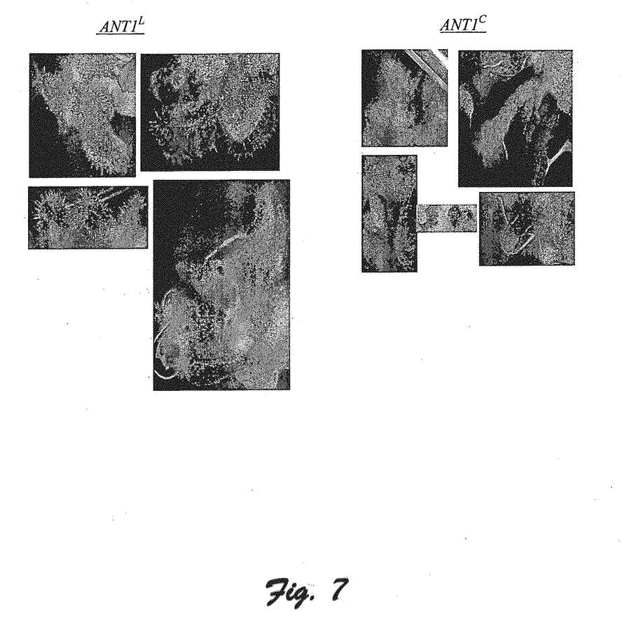

Reference is now made to FIG. 7, presenting a photographic comparison between tobacco regenerants transformed with the ANT1 gene originating from S. chilense (ANT1^{C}) and S. lycopersicum (ANT1^{L}) under the control of cauliflower mosaic virus 35S constitutive promoter.

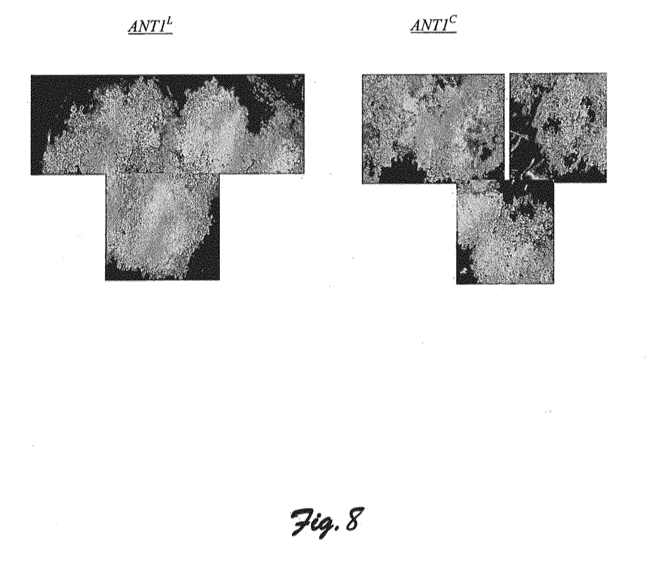

Reference is now made to FIG. 8, presenting a photographic comparison between tomato (cv. Moneymaker) regenerants transformed with the ANT1 gene originating from S. chilense (ANT1^{C}) and S. lycopersicum (ANT1^{C}) under the control of cauliflower mosaic virus 35S constitutive promoter.

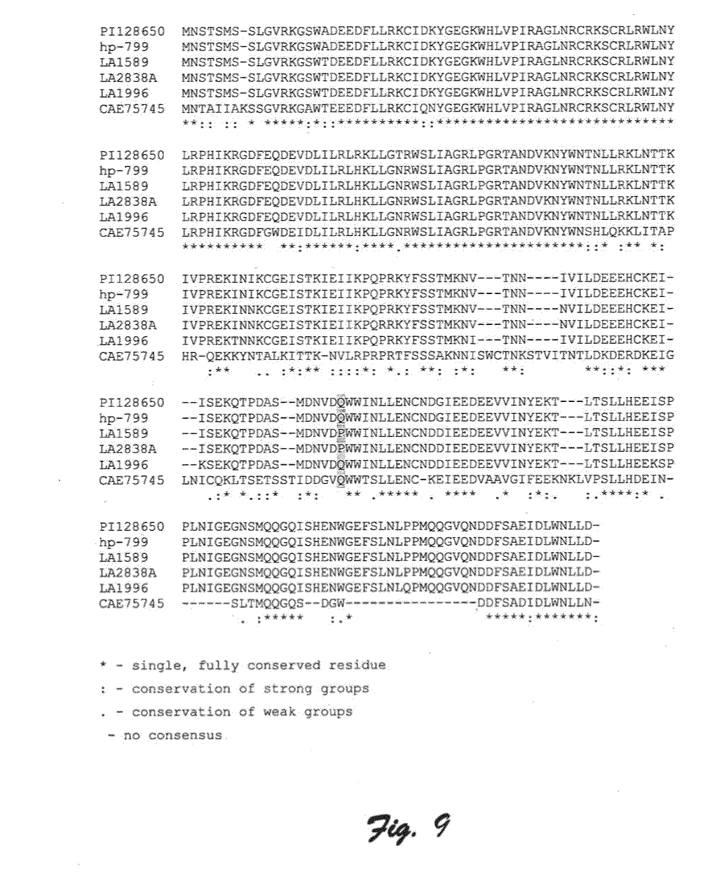

Reference is now made to FIG. 9, presenting an amino-acid alignment of the ANT1 gene cloned from tomato accessions and pepper (accessions that do not accumulate fruit anthocyanins: LA1589 is S. pimpinellifolium, LA2838A is S. lycopersicum; accessions that do accumulate fruit anthocyanins: P1128650 is S. peruvianum, hp-799 is a selection line originating from a cross between an unknown S. peruvianum and S. lycopersicum, LA1996 is AFT genotype originating from S. chilense, CAE75745 is anthocyanin accumulating pepper).

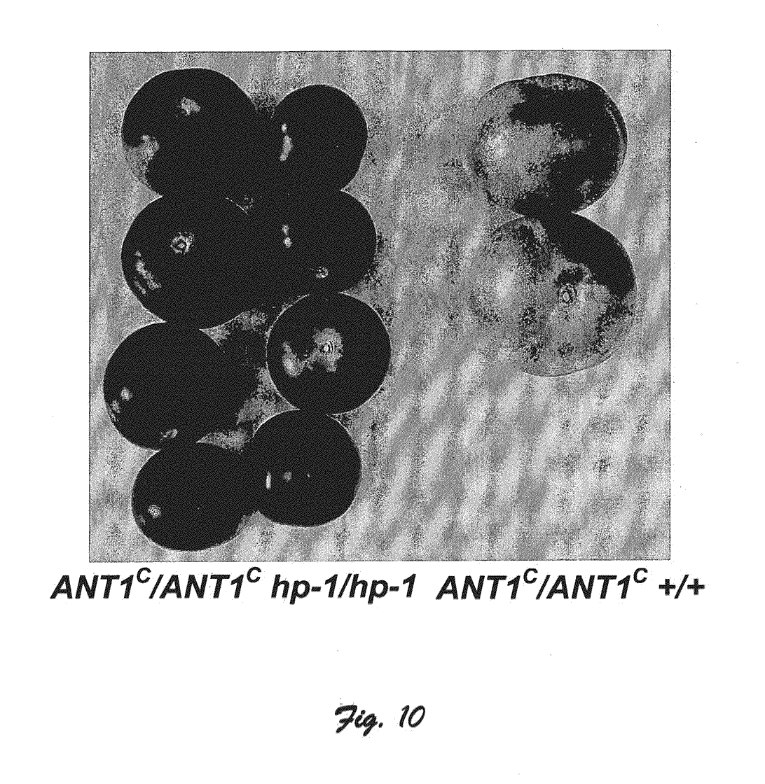

Reference is now made to FIG. 10, representing tomato fruits harvested from LA1996 plant (ANT1^{C}/ANT1^{C}+/+) and F₃ plants homozygous for both the hp-1 mutation and the ANT1^{C} allele (ANT1^{C}/ANT1^{C} hp-1/hp-1).

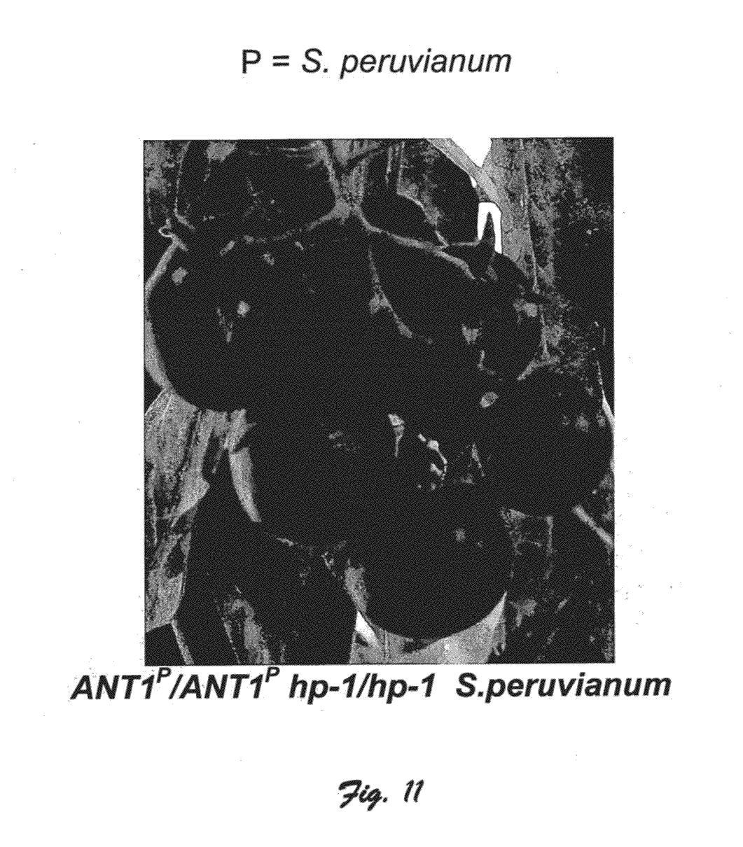

Reference is now made to FIG. 11 representing a tomato plant and fruits of an accession that is homozygous for the hp-1 mutation and the ANT1 allele originating from S. peruvianum (ANT1^{P}/ANT1^{P} hp-1/hp-1).

### Example 1

Fruits homozygous at the AFT locus contain increased levels of the flavonols quercetin and kaempherol in addition to anthocyanins. Plants of AFT genotype LA1996, red-fruited Moneymaker plants, and F₁ plants of a cross between Moneymaker and LA1996 were grown in an open field randomized-block design. Five seedlings of each genotype were planted in each of 3 blocks. Fruits were sampled at the ripe-red stage and subjected for high-performance liquid chromatography analysis to determine the levels of flavonols and anthocyanins in fruit peel. Major anthocyanins identified in ripe-red fruits and their average concentrations are presented, according to genotype, in table 3. Major flavonols present in ripe-red fruit of the same genotypes and their average concentrations are presented in table 4.

Results presented in table 3 demonstrate a statistically significant accumulation of the anthocyanins delphinidin, petunidin and malvidin in the peel of mature fruits harvested from AFT/AFT plants compared to the red-fruited Moneymaker plants. These results confirm earlier results that compared anthocyanin levels in fruits of the same AFT/AFT plants and the red-fruited processing type tomato plants UC82B (Jones et al., (2003) J Hered. 94: 449-456). In addition, results presented in table 4 show that fruits of the AFT/AFT mutant plants characterized also by a statistically significant accumulation of functional flavonols, in particular: quercetin and kaempherol. Quercetin concentration was found to be ˜3.6-fold higher in mature fruits of the AFT/AFT genotype compared to those of red-fruited Moneymaker plants based on skin weight (gFW), and ˜4.3-fold higher based on skin area (cm²). Kaempferol concentration was found to be ˜2.7-fold higher in mature fruits of the AFT/AFT genotype compared to those of red-fruited Moneymaker plants based on skin weight (gFW), and ˜3.3-fold higher based on skin area (cm²).

TABLE 3
Average anthocyanin concentration in ripe-red fruit
of LA1996 (AFT/AFT), Money maker (+/+) and their
F₁ plants (AFT/+) [(values represent peek area per g of
fresh skin weight (a) or per cm² of skin area (b)]

| | Major anthocyanins detected | | |
|---|---|---|---|
| | Malvidin | Petunidin | Delphinidin |
| Genotype | (Mean ± S.E.) | (Mean ± S.E.) | (Mean ± S.E.) |

(a) Per skin weight

| | | | |
|---|---|---|---|
| AFT/AFT | 447.5^{A} ± 91.6 | 1652.5^{A} ± 290.2 | 184.8^{A} ± 58.4 |
| AFT/+ | 27.3^{B} ± 15.8 | 107.2^{B} ± 57.3 | 12.3^{B} ± 5.7 |
| +/+ | 0^{B} ± 0 | 0^{B} ± 0 | 0^{B} ± 0 |
| p(F) | 0.008 | 0.005 | 0.042 |

(b) Per skin area

| | | | |
|---|---|---|---|
| AFT/AFT | 11.7^{A} ± 3.5 | 44.0^{A} ± 11.4 | 5.2^{A} ± 1.8 |
| AFT/+ | 1.8^{B} ± 1.1 | 7.3^{B} ± 4.4 | 0.7^{B} ± 0.3 |
| +/+ | 0^{B} ± 0 | 0^{B} ± 0 | 0^{B} ± 0 |
| p(F) | 0.033 | 0.025 | 0.070 |

Different superscript letters represent statistically significant difference between means within each metabolite.

TABLE 4
Average flavonol concentration in ripe-red fruit of
LA1996 (AFT/AFT), Money maker (+/+) and their F₁
plants (AFT/+) [(values represent peek area per g of
fresh skin weight (a) or per cm² of skin area (b)].

| | Major flavonoids detected | | |
|---|---|---|---|
| | Naringenin | Quercetin | Kaempferol |
| Genotype | (Mean ± S.E.) | (Mean ± S.E.) | (Mean ± S.E.) |

(a) Per skin weight

| | | | |
|---|---|---|---|
| AFT/AFT | 4443.0^{A} ± 836.9 | 18440.2^{A} ± 2210.3 | 1124.7^{A} ± 170.8 |
| AFT/+ | 2093.0^{A} ± 574.9 | 7366.2^{B} ± 1598.7 | 478.3^{B} ± 66.0 |
| +/+ | 3393.0^{A} ± 908.0 | 5156.0^{B} ± 623.1 | 411.5^{B} ± 21.2 |
| p(F) | 0.085 | 0.001 | 0.006 |

(b) Per skin area

| | | | |
|---|---|---|---|
| AFT/AFT | 127.8^{A} ± 25.6 | 523.5^{A} ± 52.8 | 32.2^{A} ± 4.8 |
| AFT/+ | 62.3^{A} ± 17.4 | 221.3^{B} ± 50.4 | 14.3^{B} ± 2.1 |
| +/+ | 81.7^{A} ± 25.0 | 120.2^{B} ± 17.2 | 9.6^{B} ± 0.9 |
| p(F) | 0.092 | 0.002 | 0.008 |

Different superscript letters represent statistically significant difference between means within each metabolite.

Results presented in tables 3 and 4 show that anthocyanin and flavonol concentrations in the fruit skins heterozygous F₁ plants are usually higher compared to red-fruited genotype, but much lower than the LA1996 genotype (AFT/AFT). These results indicate a partially dominant effect of the AFT gene (or genes).

Our statistical analysis, however, failed to reveal statistically significant differences between the average anthocyanin and flavonol concentration in fruits obtained from F₁ plants when compared to their red-fruited Moneymaker counterparts).

### Example 2

AFT plants are characterized by transcriptional up-regulation of key enzymes of the flavonid biosynthetic pathway. RNA samples for real-time PCR were extracted from mature-green fruits harvested from LA1996 plants and the two red-fruited genotypes: VF36 and Rutgers, planted within the framework of the preliminary experiment mentioned above. Following cDNA synthesis and real-time PCR analysis, These 3 genotypes were compared in relation to the transcriptional profile of 4 structural enzymes of the flavonoid biosynthetic pathway-CH1, CH2, F3H, and DFR (primers shown in Table 3). Results indicate an extreme up-regulation of CHS1, CHS2, and DFR and a moderate down-regulation of F3H in the LA1996 when compared to the two red-fruited genotypes (Data not shown). Of particular interest was the extreme up regulation observed in the two CHS genes, operating at the initial step of flavonoid biosynthesis and the DFR gene that encodes an enzyme active at a the later stages of the pathway (FIG. 1). Analyses were repeated using samples taken from the randomized-block experiment, (see example 1). Samples for real-time PCR were taken from LA1996 plants, red-fruited Moneymaker plants, and F₁ plants of the cross between these two lines from the 3 blocks mentioned above. Results showing the fold-increase in transcription of key genes of the flavonoid biosynthetic pathway are presented in Tab. 5.

TABLE 5
Fold-increase in transcription of structural genes of
the flavonoid biosynthetic enzymes in mature-green fruits
harvested from homozygous and heterozygous AFT plants
relative to red-fruited Moneymaker plants (+/+).

| | Fold increase of | Fold increase of |
|---|---|---|
| | AFT/AFT vs. +/+ | AFT/+ vs. +/+ |
| Gene | (Mean ± S.E.) | (Mean ± S.E.) |
| CHS1 | 62.0* ± 20.4 | 208.8* ± 64.9 |
| CHS2 | 3.9* ± 1.0 | 12.1* ± 3.1 |
| CHI | 0.6^{NS} ± 0.2 | 1.3^{NS} ± 0.4 |
| DFR | 176.2* ± 34.4 | 496.1* ± 147.4 |

*Indicates that the fold increase is statistically different from the value of 1
^{NS}Indicates that the fold increase is not statistically different from the value of 1

These results confirmed that the genes encoding CHS1, CHS2 and DFR are indeed substantially up-regulated in fruit-skin of both homozygous and heterozygous AFT plants compared to their red-fruited counterparts.

### Example 3

Genes encoding CHS1, CHS2 DFR and F3H are not polymorphic in AFT plants. CHS is the gene encoding the enzyme(s) operating on the first committed step in the flavonoid biosynthetic pathway. Due to their significant transcriptional up-regulation as shown above, it is hypothesized that either CHS1 or CHS2 could be the gene that causes the AFT phenotype. To examine this hypotheses locus specific primers were designed for each of these two genes (Table 1), PCR amplified the corresponding genomic regions from LA1996 and two red-fruited cultivars: VF36 (LA0490) and Rutgers (LA1090), and digested them with 31 (CHS1) and 35 (CHS2) restriction endonucleases.

No polymorphism was obtained between LA 1996 and red-fruited cultivars for these two genes. Similarly, no polymorphism was obtained for the DFR (27 restriction endonucleases) and F3H (22 restriction endonucleases) genes, operating at later stages of anthocyanin biosynthesis and that were found to be transciptionally miss-regulated in our former experiments (see herein above). These results are consistent with the proposition that a regulatory gene should be the gene that encodes the AFT phenotype.

### Example 4

The tomato ANT1 gene is a highly likely gene candidate that encodes the AFT phenotype. T-DNA activation-tagging experiments in tomato identified a MYB transcriptional regulator of anthocyanin biosynthesis, termed ANT1 that has high homology with Petunia Ant (Mathews et al., (2003) Plant Cell 15: 1689-1703).

Mutant ant1 tomato plants showed intense purple pigmentation from the very early stage of shoot formation in culture, reflecting activation of the biosynthetic pathway leading to anthocyanin accumulation. Vegetative tissues of anr1 plants displayed intense purple color, and the fruit showed purple spotting on the epidermis and pericarp. Similar to the fruit transcriptional results (example 2), ant1 mutant seedlings showed up-regulation of genes that encode proteins active at the early (CHS) and late (DFR) of anthocyanin biosynthesis (Mathews et al. (2003) Plant Cell 15: 1689-1703).

The ANT1 gene sequence was later used as a RFLP probe to show a complete co-segregation, using 295 F2 individuals, between ANT1 and the pepper A gene, a dominant gene that accumulate anthocyanin pigments in the foliage, flower and immature fruit (Borovsky et al. (2004) Theor Appl Genet. 109: 23-29). The A gene was mapped to the pepper chromosome 10, a chromosome that was earlier shown to be not polymorphic in LA1996 (Jones et al., (2003) J Hered 94: 449-456). Nonetheless, it was decided to sequence-characterize the ANT1 gene from LA1996 and the red fruited cv. Ailsa Craig plants to detect possible nucleotide polymorphisms that would underline the ANT1 gene as a possible candidate gene for the AFT phenotype. Sequence analysis revealed multiple nucleotide differences between the two genotypes in both coding and non-coding regions of the ANT1 gene (FIG. 2). Noteworthily, a complete sequence identity was found between the nucleotide sequences of the open reading frame of cv. Ailsa Craig and the ANT1 gene sequence originally obtained from a Micro-Tom line (GenBank accession AY348870 retrieved from http://www.ncbi.nlm.nih.gov/).

Comparison of the amino acid sequence between cv. Ailsa Craig and LA1996 revealed 8 amino acids differences between the two genotypes (FIG. 3). Obviously, a complete identity was found between the amino acid sequence of cv. Ailsa Craig and the amino acid sequence of ANT1 originally cloned from a Micro-Tom line (GenBank accession AAQ55181 retrieved from http://www.ncbi.nlm.nih.gov/). Seven of the amino acids that differ between LA1996 and the two red fruited genotypes (Ailsa Craig and Micro-Tom) can be regarded as major differences (Table 6).

TABLE 6
Differences in amino acids (AA) obtained between red fruited lines
and LA1996 (the properties of each amino acid are in parenthesis).

| AA | | |
|---|---|---|
| posi- | | |
| tion | AA in red fruited | AA in LA1996 |
| 126 | Isoleucine (neutral hydrophobic) | Threonine (neutral polar) |
| 144 | Arginine (basic) | Proline (neutral hydrophobic) |
| 155 | Valine(neutral hydrophobic) | Isoleucine (neutral hydrophobic) |
| 159 | Asparagine (neutral polar) | Isoleucine (neutral hydrophobic) |
| 172 | Isoleucine (neutral hydrophobic) | Lysine (basic) |
| 187 | Proline (neutral hydrophobic) | Glutamine (neutral polar) |
| 222 | Isoleucine (neutral hydrophobic) | Lysine (basic) |
| 252 | Proline (neutral hydrophobic) | Glutamine (neutral polar) |

Based on the nucleotide sequence differences between LA1996 and the red-fruited genotypes in the ANT1 gene, PCR primers were designed (Table 1) that were successfully used in PCR amplification reaction. Amplification products were digested with NcoI restriction endonuclease, to show codominant polymorphisms between the ANT1 alleles originating from S. lycopersicum (ANT1^{L}) and from S. chilense (ANT1^{C}) as shown in FIG. 4. In addition to the nucleotide and protein sequence polymorphism elaborated above, the ANT1 gene showed a statistically significant 4.9-fold (S.E. is 1.4) transcriptional down-regulation in tomato peel taken from fruits harvested from LA1996 compared to the red-fruited Money maker counterparts. A statistically significant transcriptional down-regulation was also observed in fruits harvested from F₁ plants resulting from a cross between Moneymaker and LA 1996, but the fold-reduction in transcription was halved (2.5±0.2).

### Example 5

The tomato ANT1 gene is highly associated with the trait of anthocyanin accumulation in the tomato fruit. A linkage analysis was made to determine whether ANT1 and the trait of anthocyanin accumulation are linked. For this purpose An F₂ population resulting from a cross between LA1996 and cv. Ailsa Craig, homozygous for the hp-1 mutation was generated. The hp-1/hp-1 mutant plants shown to have increased flavonoid accumulation in ripe-red homozygous hp mutant plants (Yen et al., (1997) Theor Appl Genet. 95: 1069-1079; Bino et al., (2005) New Phytologist 166: 427-438, Levin et al., (2006) Israel J of Plant Sci, in press) were used, on the hypothesis that aggregation of anthocyanin accumulation may be observed in hp-1/hp-1 mutant plants that also carry the AFT gene. A total of 247 F₂ plants were genotyped for both the HP-1 and ANT1 genes using the pyrosequencing primers designed earlier (Lieberman et al., (2004) Theor Appl Genet. 108: 1574-1581), and PCR primers presented in table 1, respectively. The trait of anthocyanin accumulation was recorded by visual inspection of mature-green and ripe-red fruits.

TABLE 7
Association between the ANT1 gene and that trait of anthocyanin
accumulation in F₂ population segregating for ANT1 and hp-1
(upper raw-number of plants screened and in parentheses-
number of plants showing anthocyanin accumulation.

| | ANT1 Genotype | | |
|---|---|---|---|
| hp-1 genotypes | ANT1^{C}/ANT1^{C} | ANT1^{C}/ANT1^{L} | ANT1^{L}/ANT1^{L} |
| hp-1/hp-1 | 23 | 40 | 9 |
| | (23) | (40) | (0) |
| hp-1/+ | 33 | 63 | 23 |
| | (33) | (61) | (0) |
| +1+ | 18 | 30 | 8 |
| | (18) | (28) | (0) |

Results presented in table 7 and FIG. 5 show a strong association between the ANT1^{C} and the trait of anthocyanin accumulation with a noteworthy complete association within homozygous hp-1/hp-1 genotypes. Nonetheless, 4 heterozygous ANT1^{C}/ANT1^{L} plants failed to show a phenotype anthocyanin accumulation in the mature-green or ripe-red fruits as would be expected assuming ANT1^{C} is dominant over ANT1^{L}. Regarded as recombinants, these plants should point to ˜0.8 ceniMorgan distance between the ANT1 and AFT genes (calculated on F₂ basis). We however dispute this claim because, in our growth conditions, we at times could not observe any visible phenotype in heterozygous ANT1^{C}/ANT1^{L} plants resulting from crosses between LA1996 and several red fruited open-pollinated cultivars, including Ailsa Craig. The inability of heterozygous plants to display phenotypes is also well demonstrated in our metabolomics data presented in Tables 3 and 4, showing that the average anthocyanin and flavonol content in fruits harvested from F₁ ANT1^{c}/ANT1^{L} plants is much similar to their homozygous ANT1^{L}/ANT1^{L} than to their homozygous ANT1^{C}/ANT1^{C} counterparts. To further validate our claim of a possible complete linkage between ANT1 and AFT, the 2 non-hp-1 heterozygous ANT1^{c}/ANT1^{L} F₂ plants that did not display the characteristic phenotype of the AFT phenotype were allowed to self pollinate. Sixty plants of each of the resulting F₃ populations were planted. Visual inspection of their fruits upon ripening revealed that these two populations indeed segregate for the AFT trait as would be expected from heterozygous plants. In addition, a plant homozygous for the hp-1 mutation and heterozygous for the ANT1 gene (hp-1/hp-1 ANT1^{C}/ANT1^{L}) was allowed to self-pollinate and the resulting F₃ plants were genotyped for the ANT1 gene. Eighteen plants representing each of the resulting genotypes were planted. Upon fruit maturation, these plants were visually inspected and a complete association was found between the ANT1 genotype and the AFT phenotype, again demonstrating a strong association and possibly a complete linkage between the ANT1 and the AFT genes.

### Example 6

The tomato AFT gene maps to chromosome 10. The strong association between the AFT gene, introgressed from LA1996, and the ANT1 gene sequence allows the chromosomal location of the AFT gene to be mapped onto the tomato genome for the first time. S. pennellii introgression lines were used for that purpose (Eshed et al., (1992) Theor Appl Genet. 83: 1027-1034). Results summarized in FIG. 6 show that the ANT1 is mapped to the longer arm of the tomato chromosome 10, exclusively to introgression line 10-3. The strong association obtained in this study between ANT1 and AFT trait indicates that the gene that causes the AFT phenotype is also localized to the long arm of the tomato chromosome 10.

### Example 7

The hp-1 mutation exaggerates anthocyanin and flavonol expression of the ANT1^{C} allele in a more than additive manner. As visually displayed in FIG. 1 the hp-1 mutation exaggerates anthocyanin expression in ripe-red fruits, attributed by the ANT1^{C} allele. This positive contribution of hp-1 can be clearly observed in homozygous ANT1^{C}/ANT1^{C} and heterozygous ANT1^{C}/ANT1^{L} plants. To quantitate this synergistic effect on fruit anthocyanin and possibly on flavonol levels mature red fruits were harvested from the 18 plants of each of the following F₃ genotypes: AFT/AFT hp-1/hp-1, AFT/+hp-1/hp-1 and ++hp-1/hp-1 as well as their initial parental lines: AFT/AFT++ (LA1996) and ++hp-1/hp-1 (cv. Ailsa Craig homozygous for the hp-1 mutation).

TABLE 8
Average concentrations of major anthocyanins detected in ripe-red
fruits of parental and F₃ genotypes [(values represent peek area per
g of fresh skin weight (a) or per cm² of skin area (b)]

| | Major anthocyanins detected | | |
|---|---|---|---|
| | Delphinidin | Petunidin | Malvidin |
| Genotype | (Mean ± S.E.) | (Mean ± S.E.) | (Mean ± S.E.) |

(a) per skin weight

| | | | |
|---|---|---|---|
| P₁(AFT/AFT, +/+) | 10.5^{B} ± 3.5 | 157.5^{B} ± 18.5 | 34.5^{B} ± 1.5 |
| P₂ (+/+, hp-1/hp-1) | 6.5^{B} ± 4.5 | 943.5^{B} ± 856.5 | 26.0^{B} ± 3.0 |
| F₃ (AFT/AFT, hp-1/hp-1) | 569.0^{A} ± 139.0 | 5766.0^{A} ± 1330.0 | 1162.0^{A} ± 270 |
| F₃ (AFT/+, hp-1/hp-1) | 95.6^{B} ± 13.5 | 1111.0^{B} ± 122 | 289.4^{B} ± 64.8 |
| F₃ (+/+, hp-1/hp-1) | 0.0^{B} ± 0.0 | 0.0^{B} ± 0 | 0.0^{B} ± 0.0 |
| p(F) | 0.0006 | 0.0005 | 0.0005 |

(b) per skin area

| | | | |
|---|---|---|---|
| P₁(AFT/AFT, +/+) | 0.21^{B} ± 0.05 | 3.20^{B} ± 0.00 | 0.72^{B} ± 0.12 |
| P₂ (+/+, hp-1/hp-1) | 0.13^{B} ± 0.09 | 19.00^{B} ± 17.00 | 0.53^{B} ± 0.07 |
| F₃ (AFT/AFT, hp-1/hp-1) | 15.40^{A} ± 3.50 | 157.60^{A} ± 33.80 | 31.80^{A} ± 6.90 |
| F₃ (AFT/+, hp-1/hp-1) | 2.10^{B} ± 0.40 | 23.60^{B} ± 3.00 | 5.80^{B} ± 1.40 |
| F₃ (+/+, hp-1/hp-1) | 0.00^{B} ± 0.00 | 0.00^{B} ± 0.0 | 0.00^{B} ± 0.00 |
| p(F) | 0.0003 | 0.0002 | 0.0002 |

Different superscript letters represent statistically significant difference between means within each metabolite.

Results presented in Table 8 show that the composite genotype AFT/AFT hp-1/hp-1 displays a significant more-than-additive effect on the anthocyanines delphinidin, petunidin and malvidin in comparison to its initial parental lines.

This genotype exhibited a similar tendency of increased levels of the flavonols quercetin and Kaemferol as displayed in table 9.

TABLE 9
Average concentrations of major flavonols detected in ripe-red
fruits of parental and F₃ genotypes [(values represent peek area per
g of fresh skin weight (a) or per cm² of skin area (b)]

| | Major flavonols detected | | |
|---|---|---|---|
| | Quercetin | Kaempferol | Naringenin |
| Genotype | (Mean ± S.E.) | (Mean ± S.E.) | (Mean ± S.E.) |

a. per skin weight

| | | | |
|---|---|---|---|
| P₁(AFT/AFT, +/+) | 6372.0^{D} ± 666.5 | 1001.0^{C} ± 32.0 | 12563.0^{B} ± 1191.0 |
| P₂ (+/+, hp-1/hp-1) | 21962.0^{BC} ± 3875.0 | 1371.0^{BC} ± 167.0 | 13828^{AB} ± 67.5 |
| F₃ (AFT/AFT, hp-1/hp-1) | 34016.0^{A} ± 6778.0 | 2598.0^{A} ± 519.7 | 13367.0^{AB} ± 2458.0 |
| F₃ (AFT/+, hp-1/hp-1) | 26528.0^{AB} ± 2298.0 | 1928.0^{AB} ± 278.9 | 18898.0^{A} ± 3364.0 |
| F₃ (+/+, hp-1/hp-1) | 13413.0^{CD} ± 4415.0 | 791.2^{C} ± 209.4 | 10517.0^{B} ± 1458.0 |
| p(F) | 0.0216 | 0.0187 | 0.2136 |

b. per skin area

| | | | |
|---|---|---|---|
| P₁(AFT/AFT, +/+) | 129.0^{D} ± 1.0 | 21.0^{C} ± 3.0 | 255.0^{B} ± 5.0 |
| P₂ (+/+, hp-1/hp-1) | 438.5^{BC} ± 77.5 | 27.5^{BC} ± 3.5 | 276.0^{AB} ± 1.0 |
| F₃ (AFT/AFT, hp-1/hp-1) | 923.8^{A} ± 143.1 | 71.0^{A} ± 12.8 | 370.6^{AB} ± 60.5 |
| F₃ (AFT/+, hp-1/hp-1) | 554.4^{B} ± 41.8 | 40.4^{B} ± 6 | 394.0^{A} ± 67.7 |
| F₃ (+/+, hp-1/hp-1) | 280.0^{CD} ± 69.7 | 17.6^{C} ± 3.2 | 243.6^{B} ± 34.0 |
| p(F) | 0.0008 | 0.0028 | 0.2503 |

Different superscript letters represent statistically significant difference between means within each metabolite.

### Example 8

Transformation of tobacco and tomato plants shows a much greater effect of ANT1^{C} anthocyanin accumulation. Transformation of AND gene originating from S. chilense (ANT1^{C}) and S. lycopersicum (ANT1^{L}) under the control of cauliflower mosaic virus 35S constitutive promoter displayed a much greater and earlier anthocyanin production in tomato and tobacco regenerants (FIGS. 7 and 8). These results underline that ANT1 is most probably the gene that encodes the AFT phenotype and that the ANT1^{C} allele has a much greater effect on anthocyanin production in comparison to the ANT1^{L} allele originating from the cultivated tomato.

### Example 9

A substitution of proline¹⁸⁷ to glutamine in the ANT1 gene—a major determinant of anthocyanin accumulation in the AFT genotype. The amino-acid sequences of the ANT1 gene cloned from high fruit anthocyanin tomato species, as well as pepper, were compared to low fruit anthocyanin tomato and pepper species. Several of these comparisons are presented in FIG. 9 and point to a substitution of proline¹⁸⁷ to glutamine in the ANT1 gene as the only amino acid that clearly differentiates between species that accumulate high concentrations of fruit anthocyanin to those that do not. This result suggests that this single amino-acid substitution alone may account for the increased fruit anthocyanin accumulation observed in AFT phenotypes. However other amino-acid changes in the ANT1 gene may generate a similar or more enhanced fruit anthocyanin accumulation phenotype.

1. An AFT gene encoding a protein characterized by at least 80% identity with the amino acid sequence shown in FIG. 9 (LA1996; SEQ ID NO:37) having been genetically introgressed into cultivated plants or elite lines, conferring higher concentrations of flavonoids on said plants as compared with prior art cultivated plants that were not introgressed with said gene.

2. The AFT gene according to claim 1, having been genetically introgressed into cultivated S. lycopersicum tomato plants or elite lines, conferring higher concentrations of flavonoids on said plants as compared with prior art cultivated S. lycopersicum plants that were not introgressed with said gene.

3. The AFT gene according to claim 1, originating from an S. chilense genotype having been genetically introgressed into cultivated S. lycopersicum tomato plants or elite lines, conferring higher concentrations of flavonoids on said plants as compared with prior art cultivated S. lycopersicum plants that were not introgressed with said gene.

4. The AFT gene according to claim 1, wherein at least a portion of the flavonoids are anthocyanins and/or flavonols.

5. The AFT gene according to claim 1, wherein said gene originates from S. peruvianum.

6. The AFT gene according to claim 1, wherein said gene is selected from a group consisting of S. habrochaites, S. cheesmaniae, S. lycoperisiciodes, S. peruvianum and S. pennelli v. puberelum.

7. An AFT S. chilense genotype introgressively-derived tomato plant, wherein said plant is characterized by high concentrations of flavonoids as compared with prior art cultivated S. lycopersicum tomato plants that were not introgressed with said genotype.

8. The tomato plant according to claim 7, wherein said plant is characterized by high concentrations of anthocyanins and/or flavonols as compared with prior art cultivated S. lycopersicum tomato plants that were not introgressed with said genotype.

9. The tomato plant according to claim 7, wherein said AFT genotype is introgressed from S. peruvianum.

10. The tomato plant according to claim 7, wherein said AFT genotype is introgressed from a group consisting of S. habrochaites, S. cheesmaniae, S. lycoperisiciodes, S. peruvianum and S. pennelli v. puberelum.

11. The tomato plant according to claim 7, obtained introgressively by a method comprising:
(i) crossing between hp-1/hp-1 accessions line tomatoes, especially S. lycopersicum, and tomatoes containing AFT gene from S. chilense;
(ii) selfing F₁ plants resulting from said cross;
(iii) generating an F₂ population segregating for both the hp-1 mutation and the AFT allele;
(iv) selecting F₂ plants homozygous for the hp-1 mutation and the AFT locus from S. chilense;
(v) selfing said F₂ plants to generate an F₃ population and further populations (F₄, F₅ and so forth) so as to obtain a pure-bred parental line characterized by high levels of flavonoids in a more than additive manner as compared with prior art cultivated S. lycopersicum tomato plants and/or initial parental lines; and,
(vi) using this parental line in crosses with other similar or different parental lines to obtain commercial F₁ hydbrids.

12. The tomato plant according to claim 11, wherein said flavonoids include anthocyanins and flavonols, especially delphinidin, petunidin, malvidin, quercetin, kaempferol and naringenin.

13. The tomato plant according to claim 11, obtained introgressively by a method of crossing wherein at least one parental is a high pigment accession line especially S. lycopersicum, said high pigment allelle selected from a group characterized by one or more homozygotic alleles defined as hp-1, hp-1^{w}, hp-2, hp-2^{j}, hp-2^{dg}, such tomato plants crossed with plants containing AFT gene from S. chilense.

14. The tomato plant according to claim 11, obtained introgressively by a method of crossing wherein at least one parental is a high pigment accession line especially S. lycopersicum, said high pigment allelle selected from a group characterized by one or more homozygotic alleles at the UV-DAMAGED DNA BINDING PROTEIN 1 (DDB1) or DEETIOLATED 1 (DET 1) genes, such as: hp-1, hp-1^{w}, hp-2, hp-2^{j}, hp-2^{dg}, such tomato plants crossed with plants containing AFT gene from S. chilense.

15. The tomato plant according to claim 11, obtained introgressively by a method of crossing wherein at least one parental is a high pigment accession line especially S. lycopersicum, said high pigment allelle selected from a group characterized by one or more homozygotic alleles at photomorphogenic genes isophenotypic to hp-1, hp-1^{w}, hp-2, hp-2^{j}, hp-2^{dg} mutant plants defective at the UV-DAMAGED DNA BINDING PROTEIN 1 (DDB1) or DEETIOLATED 1 (DET1) genes, such tomato plants crossed with plants containing AFT gene from S. chilense.

16. The tomato plant obtained according to claim 11, additionally comprising a step of selecting an F₂ plant homozygous at the AFT locus originating from S. chilense by means of a DNA marker.

17. The tomato plant obtained according to claim 11, wherein said AFT genotype originates from S. peruvianum.

18. The tomato plant obtained according to claim 11, wherein said AFT genotype is selected from a group consisting of S. habrochaites, S. cheesmaniae, S. lycoperisiciodes, S. peruvianum and S. pennelli v. puberelum.

19. The tomato plant obtained according to claim 16, wherein said DNA marker originating from S. peruvianum.

20. The tomato plant obtained according to claim 16, wherein said DNA marker originating from a group consisting of S. habrochaites, S. cheesmaniae, S. lycoperisiciodes, S. peruvianum and S. pennelli v. puberelum.

21. The tomato plant according to claim 11, wherein said flavonoid is selected from any member of a group consisting of the flavonoid aglycones, flavonoid O-glycosides, flavonoid C-glycosides, flavonoids with hydroxyl and/or methoxy substitutions, C-methylflavonoids, methylenedioxy flavonoids chalcones, aurones, dihydrochalcones, flavanones, dihydroflavanols, anthclors, proanthocyanidins, condensed proanthocyanidins, leucoanthocyanidins, flavan-3,4-ols, flavan-3-ols, glycosylflavonoids, biflavonoids, triflavonoids, isoflavonoids, isoflavones, isoflavanones, rotenonoids, pterocarpans, isoflavans, quinone derivatives, 3-aryl-4-hydroxycoumarins, 3-arylcoumarin, isoflav-3-enes, coumestans, α-methyldeoxybenzoins, 2-arylbenzofurans, isoflavanol, and coumaronochromone.

22. The tomato plant according to claim 8, wherein said anthocyanin is selected from a group consisting of delphidin, petundin, and malvidin.

23. The tomato plant according to claim 1, wherein said flavonoid is selected from a group consisting of quercetin and kaempherol.

24. The tomato plant according to claim 11, wherein said flavonoid is selected from a group consisting pf 4,2,4,6-tetrahydroxychalcone, naringenin, kaempherol, dihydroxy kaempherol, myrecetin, quercetin, dihydroquercetin, dihydromyrecetin, leucopelargonidin, leucocyanidin, leucodelphinidin, pelargonidin-3-glucoside, cyanidin-3-glucoside and delphinidin-3-glucoside.

25. The tomato plant according to claim 11, wherein said flavonoid is selected from a (i) group consisting of secondary plant metabolites derived from the 2-phenylchromone (2-phenyl-1,4-benzopyrone) structure; (ii) isoflavonoids, wherein said metabolites are derived from the 3-phenylchromone (3-phenyl-1,4 benzopyrone) structure; and, (iii) neoflavonoids wherein said metabolites are derived from the 4-phenylcoumarine (4-phenyl-1,2-benzopyrone) structure.

26. A DNA sequence which encodes for a protein characterized by at least 80% homology with the amino acid sequence shown in FIG. 9 (LA1996; SEQ ID NO:37) providing high flavonoid concentrations in tomato plants as compared with prior art cultivated S. lycopersicum tomato plants.

27. A DNA sequence according to claim 26, characterized by at least 80% homology with the nucleic acid sequence shown in the lower row of FIG. 2 (LA1996; SEQ ID NO:30) from residue 1 to residue 1008, providing high flavonoid concentrations in tomato plants as compared with prior art cultivated S. lycopersicum tomato plants.

28. A DNA sequence according to claim 26, wherein said DNA confers accumulation or expression of metabolites of the flavonoid pathway, especially anthocyanin or flavonols, in plants, plant parts or seeds thereof.

29. A DNA sequence according to claim 26, wherein said DNA confers accumulation or expression of metabolites of the flavonoid pathway, especially anthocyanin or flavonols, in tomato plants, especially S. lycopersicum, plant parts or seeds thereof.

30. A DNA sequence according to claim 27, found useful in screening germ plasm, seeds, seedlings, cali, plants or plant parts for introgression of the AFT genotype in cultivated tomato accessions wherein said DNA is characterized by at least 80% homology with the nucleic acid sequence shown in the lower row of FIG. 2 (LA1996; SEQ ID NO:30) from residue 1 to residue 1008.

31. A transgenic plant expressing metabolites of the flavonoid pathway, especially anthocyanin or flavonols, in plants, plant parts or seeds thereof, said plant comprising DNA with at least 80% homology with the nucleic acid sequence shown in the lower row of FIG. 2 (LA1996; SEQ ID NO:30) from residue 1 to residue 1008; said DNA recombined into a plurality of one or more transformation and/or expression vectors, useful for transformation and/or expression in plants.

32. A method of obtaining an AFT gene encoding a protein characterized by at least 80% identity with the amino acid sequence shown in FIG. 9 (LA1996; SEQ ID NO:37) having been genetically introgressed into cultivated plants or elite lines, conferring higher concentrations of flavonoids on said plants as compared with prior art cultivated plants that were not introgressed with said gene.

33. A method of obtaining AFT S. chilense genotype introgressively-derived tomato plants, characterized by high concentrations of anthocyanins and/or flavonoids as compared with prior art cultivated S. lycopersicum tomato plants; said method comprising:
(i) crossing between hp-1/hp-1 accessions line tomatoes, especially S. lycopersicum, and tomatoes containing AFT gene from S. chilense;
(ii) selfing F₁ plants resulting from said cross;
(iii) generating an F₂ population segregating for both the hp-1 mutation and the AFT allele;
(iv) selecting F₂ plants homozygous for the hp-1 mutation and the AFT locus from S. chilense;
(v) selfing said F₂ plants to generate an F₃ population so as to obtain a pure-bred parental line characterized by high levels of flavonoids in a more than additive manner as compared with prior art cultivated S. lycopersicum tomato plants and/or initial parental lines; and,
(v) using this parental line in crosses with other similar or different parental lines to obtain commercial F₁ hydbrids.

34. A method of obtaining AFT S. chilense genotype introgressively-derived tomato plants, characterized by high concentrations of anthocyanins and/or flavonoids as compared with prior art cultivated S. lycopersicum tomato plants; said method comprising:
(i) crossing between hp-1/hp-1 accessions line tomatoes, especially S. lycopersicum, and tomatoes containing AFT gene from S. chilense;
(ii) selfing F₁ plants resulting from said cross;
(iii) generating an F₂ population segregating for both the hp-1 mutation and the AFT allele;
(iv) selecting F₂ plants homozygous for the hp-1 mutation and the AFT locus from S. chilense;
(v) selfing said F₂ plants to generate an F₃ and further populations (F₄, F₅ and so forth) so as to obtain a pure-bred parental line characterized by high levels of anthocyanins and flavonols especially delphidin, petundin, malvidin, quercetin and kaempherol, in a more than additive manner as compared with prior art cultivated S. lycopersicum tomato plants and/or initial parental lines; and,
(v) using this parental line in crosses with other similar or different parental lines to obtain commercial F₁ hydbrids.

35. The method according to claim 34, said method of obtaining introgressed plants by crossing wherein at least one parental is a high pigment accession line especially S. lycopersicum, such that said high pigment alleles are selected from a group characterized by one or more homozygotic alleles defined as hp-1^{w}, hp-2, hp-2^{j}, hp-2^{dg}, such tomato plants crossed with plants containing AFT gene from S. chilense.

36. The method according to claim 34, said method of obtaining introgressed plants by crossing wherein at least one parental is a high pigment accession line especially S. lycopersicum, such that said high pigment allelles are selected from a group characterized by one or more homozygotic alleles at the UV-DAMAGED DNA BINDING PROTEIN 1 (DDB1) or DEETIOLATED 1 (DET1) genes, such as: hp-1, hp-1^{w}, hp-2, hp-2^{j}, hp-2^{dg}, such tomato plants crossed with plants containing AFT gene from S. chilense.

37. The method according to claim 34, said method of obtaining introgressed plants by crossing wherein at least one parental is a high pigment accession line especially S. lycopersicum, such that said high pigment alleles are selected from a group characterized by one or more homozygotic alleles at photomorphogenic genes isophenotypic to hp-1, hp-1^{w}, hp-2, hp-2^{j}, hp-2^{dg} mutant plants defective at the UV-DAMAGED DNA BINDING PROTEIN 1 (DDB1) or DEETIOLATED 1 (DET1) genes, such tomato plants crossed with plants containing AFT gene from S. chilense.

38. The method according to claim 34, additionally comprising of selecting by means of a DNA marker an F₂ plant homozygous for the HP-1 and AFT loci.

39. The method according to claim 34, wherein said anthocyanin is selected from a group consisting of delphidin, petundin and malvidin.

40. The method according to claim 34, wherein said flavonoid is selected from a group consisting of quercetin, kaempherol and naringenin.

41. A method for obtaining a tomato plant with high flavonoids as compared with prior art cultivated S. lycopersicum tomato plants, according to claim 34, wherein said flavonoid is selected from a group consisting pf 4,2,4,6-tetrahydroxychalcone, naringenin, kaempherol, dihydroxy kaempherol, myrecetin, quercetin, dihydroquercetin, dihydromyrecetin, leucopelargonidin, leucocyanidin, leucodelphinidin, pelargonidin-3-glucoside, cyaniding-3-glucoside and delphinidin-3-glucoside.

42. A method for obtaining a tomato plant according to claim 34, wherein said flavonoids are selected from a group consisting of secondary plant metabolites derived from (i) 2-phenylchromone (2-phenyl-1,4-benzopyrone) structure; (ii) isoflavonoids, wherein said metabolites are derived from the 3-phenylchromone (3-phenyl-1,4 benzopyrone) structure; and, (iii) neoflavonoids wherein said metabolites are derived from the 4-phenylcoumarine (4-phenyl-1,2-benzopyrone) structure.

43. A method for obtaining a tomato plant according to claim 34, wherein said flavonoids are selected from any member of a group consisting of the flavonoid aglycones, flavonoid O-glycosides, flavonoid C-glycosides, flavonoids with hydroxyl and/or methoxy substitutions, C-methylflavonoids, methylenedioxyflavonoids methylenedioxy flavonoids chalcones, aurones, dihydrochalcones, flavanones, dihydroflavanols, anthclors, proanthocyanidins, condensed proanthocyanidins, leucoanthocyanidins, flavan-3,4-ols, flavan-3-ols, glycosylflavonoids, biflavonoids, triflavonoids, isoflavonoids, isoflavones, isoflavanones, rotenonoids, pterocarpans, isoflavans, quinone derivatives, 3-aryl-4-hydroxycoumarins, 3-arylcoumarin, isoflav-3-enes, coumestans, α-methyldeoxybenzoins, 2-arylbenzofurans, isoflavanol, and coumaronochromone.

44. A method for obtaining DNA which encodes for a protein comprising at least 80% identity with an amino acid sequence shown in FIG. 9 (LA1996; SEQ ID NO:37); said method comprising identifying and optionally verifying said encoded amino acid sequence, said sequence being of a protein naturally occurring in S. chilense responsible for the AFT phenotype and enhanced flavonoid concentration.

45. A method useful for obtaining nucleic acid characterized by at least 80% homology with the nucleic acid sequence shown in the lower row of FIG. 2 (LA1996; SEQ ID NO:30) from residue 1 to residue 1008, said method comprising identifying and optionally verifying said amino acid sequence as encoding a protein naturally occurring in S. chilense responsible in least in part for the AFT phenotype.

46. A method according to claim 34 for obtaining tomato plants high in flavonoids as compared with prior art cultivated S. lycopersicum tomato plants; facilitated by screening germ plasm, seeds, seedlings, cali or plants for introgression of the AFT S. chilense genotype into cultivated tomato accessions; said method further comprising:
(i) obtaining nucleic acid at least 80% homologous with the nucleic acid sequence shown in the lower row of FIG. 2 (LA1996; SEQ ID NO:30) from residue 1 to residue 1008;
(ii) preparing PCR primers as defined in Table 1 (SEQ ID NOS:1-10);
(iii) amplifying DNA of (i); and,
(iv) probing target tissue therewith.

47. A transgenic method for accumulating or expressing metabolites of the flavonoid pathway, especially anthocyanin or flavonols, in plants, plant parts or seeds thereof, said method comprising:
(i) obtaining DNA at least 80% homologous with the nucleic acid sequence shown in the lower row of FIG. 2 (LA1996; SEQ ID NO:30) from residue 1 to residue 1008; and,
(ii) combining said DNA into a plurality of one or more transformation and/or expression vectors, useful for transformation and/or expression in plants.

48. The transgenic method according to claim 47, especially useful for tomato plants, tomato plant parts or seeds thereof.
